(19) United States
(12) Patent Application Publication
(10) Pub. No.: US 2014/0329744 A1
(43) Pub. Date: Nov. 6, 2014

(54) STABLE NON-AQUEOUS LIQUID PHARMACEUTICAL COMPOSITIONS COMPRISING AN INSULIN

(71) Applicant: Novo Nordisk A/S, Bagsvaerd (DK)

(72) Inventors: Helle Naver, Alleroed (DK); Florian A. Foeger, Fredericksberg C (DK); Thomas Hoeg-Jensen, Klampenborg (DK); Charlotte H. Fynbo, Herlev (DK)

(21) Appl. No.: 14/333,793

(22) Filed: Jul. 17, 2014

Related U.S. Application Data
(63) Continuation of application No. 13/496,529, filed on Apr. 23, 2012.

(30) Foreign Application Priority Data
Sep. 16, 2009 (EP) 09170389.2

(51) Int. Cl. A61K 47/18; A61K 38/28

(52) U.S. Cl. CPC A61K 47/18; A61K 38/28
USPC 514/5.9

(57) ABSTRACT

The invention describes a non-aqueous liquid pharmaceutical composition comprising at least one lipid and at least one insulin. Also described is a method of producing a pharmaceutical composition comprising a lipid and a method of purifying a lipid, a cosolvent, a surfactant or a pharmaceutical composition comprising a lipid.

### CROSS-REFERENCE TO RELATED APPLICATIONS

This application is a continuation of U.S. application Ser. No. 13/496,529, filed Apr. 23, 2012, which is a 35 U.S.C. §371 national stage application of International Patent Application PCT/EP2010/063610 (published as WO 2011/033019 A1), filed Sep. 16, 2010, which claimed priority of European Patent Application 09170389.2, filed Sep. 16, 2009, the contents of which are incorporated herein by reference.

### FIELD OF THE INVENTION

The present invention is related to stable non-aqueous liquid pharmaceutical compositions comprising at least one insulin and at least one lipid. Also described are methods of producing pharmaceutical compositions comprising at least one lipid, and methods for purifying a lipid, a cosolvent, a surfactant or a pharmaceutical composition comprising a lipid.

### BACKGROUND OF THE INVENTION

Previous lipid based compositions with insulin have proven very efficient for the oral administration of insulin. However, the shelf life of these compositions is below 3 months as a result of the presence of lipid impurities and degradation products. Pharmaceutical drug development requires at least 2 years of shelf life.

Manufactured lipids, natural lipids, caprylates and surfactants may contain aldehyde and ketones in concentrations around 10-200 ppm. Furthermore the exposure of lipids to air results in oxidation and aldehyde formation. The two main insulin degradation products identified in lipid water free composition are aldehyde derived degradation products.

Aldehydes and ketones can often be tolerated in aqueous formulations in amounts up to around 200 ppm in the excipients in an aqueous pharmaceutical composition while chemical stability of insulin is retained. If the aldehydes and ketones are present above this limit degradation products such as high molecular weight polymers (HMWP) are formed (Brange et al. (1992) Pharmaceutical Research. 9:727-734)

It is known that aqueous pharmaceutical compositions can comprise e.g. ethylenediamine for stability purposes. For example WO2006125763 describes aqueous pharmaceutical polypeptide compositions comprising ethylenediamine as a buffer.

However, a method remains to be found for stabilising non-aqueous liquid insulin pharmaceutical compositions comprising one or more lipids.

It is thus the aim of the invention to provide a non-aqueous liquid pharmaceutical composition comprising a lipid and an insulin, which is chemically stabilized and thus has an acceptable shelf life. Also a method of obtaining a pharmaceutical composition comprising at least one lipid and a method for purifying the composition and/or ingredients of the composition to obtain chemical stability is provided.

### SUMMARY OF THE INVENTION

The invention is related to a non-aqueous liquid pharmaceutical composition comprising at least one lipid, at least one insulin, at least one scavenger and optionally at least one surfactant, wherein the scavenger is a nitrogen containing nucleophilic compound such as an amine, for example a diamine, a triamine, an oxyamine, a hydrazine or a hydrazide. In one aspect, the scavenger in the non-aqueous liquid pharmaceutical composition is ethylenediamine or a derivative thereof.

In one aspect, the lipid in the non-aqueous liquid pharmaceutical composition is a high purity lipid.

Also a method for purifying a lipid, a cosolvent, a surfactant or a pharmaceutical composition comprising a lipid is described, wherein purification is performed on a nitrogen containing, surfactant compatible, nucleophilic matrix whereby removal of excess aldehyde is achieved. A non-aqueous liquid pharmaceutical composition is furthermore described, wherein the lipid has been purified by said method.

### BRIEF DESCRIPTION OF THE DRAWINGS

FIG. 1: NMR spectra showing the removal of aldehydes from two different grades of Labrasol.

FIG. 2: Standard curve MBTH aldehyde analysis

FIG. 3: Stability of the derivative of insulin B29K(N(eps)Octadecanedioyl-gGlu-OEG-OEG) A14E B25H desB30 in liquid lipid for oral administration as a function of purification of lipid mix. Contents of each formulation 1-6 is shown in table 9

FIG. 4: Stability of the derivative of insulin B29K(N(eps)Octadecanedioyl-gGlu-OEG-OEG) A14E B25H desB30 dissolved in various sources of propylene glycol

FIG. 5: Stability of the derivative of insulin B29K(N(eps)Octadecanedioyl-gGlu-OEG-OEG) A14E B25H desB30 in liquid lipid formulations containing various sources of Labrasol

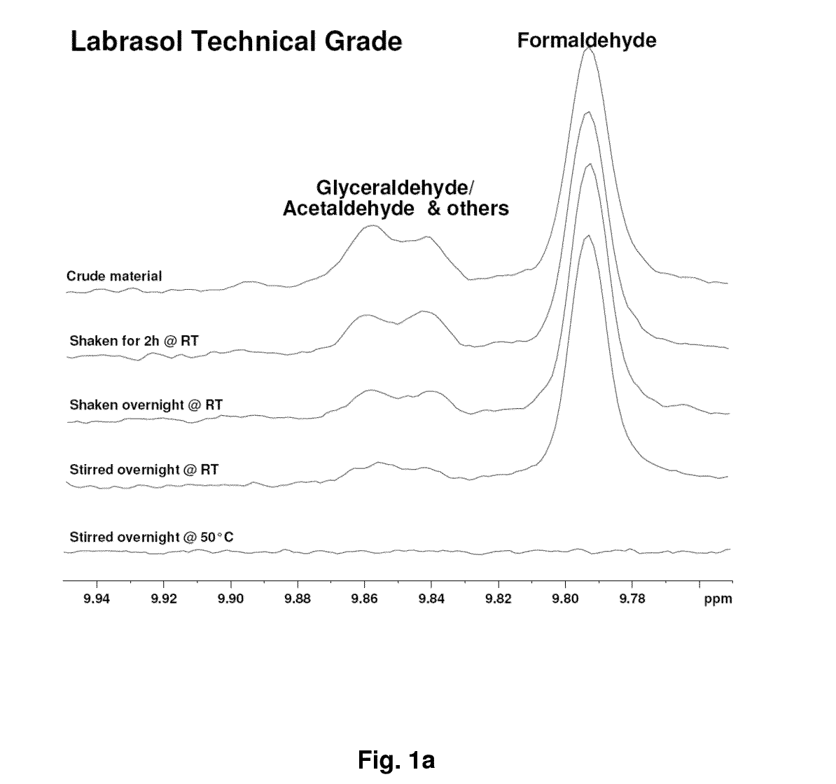

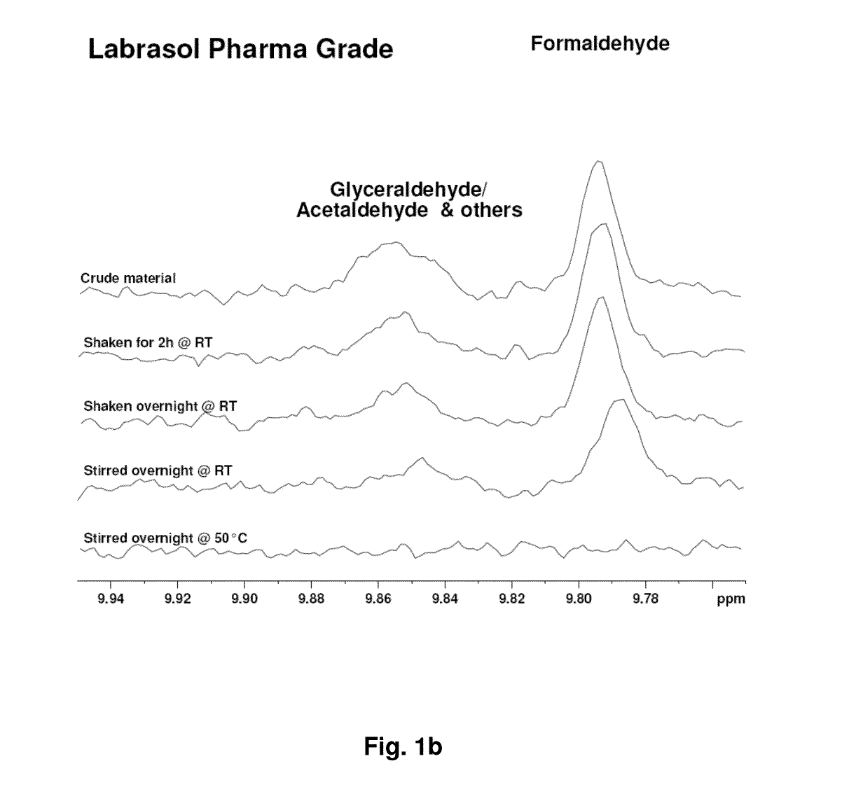

### DESCRIPTION OF THE INVENTION

It has surprisingly been found that non-aqueous liquid insulin pharmaceutical compositions comprising one or more lipids and optionally one or more surfactants can be chemically stabilized by the addition of a scavenger to the composition and/or purification of the lipid by the disclosed method.

The invention is particularly useful in large scale preparation of pharmacutical compositions where stress conditions like humidity and air are often occurring during manufacturing.

The term “scavenger” is herein used to mean a chemical substance added to the pharmaceutical composition in order to remove or inactivate reactive impurities such as aldehydes and ketones. Aldehydes and ketones may react with for example the free amino groups of insulin (A1, B1 or B29) resulting in the formation of Schiff bases which may undergo transformation to unwanted products such as e.g. insulin covalent dimers. A “scavenger” according to the invention contains a nucleophile functionality which is able to react with aldehydes and/or ketones.

In one aspect of the invention the non-aqueous insulin pharmaceutical composition further comprises a cosolvent such as e.g. propylene glycol.

In one aspect of the invention the scavenger is soluble in the cosolvent of the formulation. In one aspect of the invention the scavenger is a nitrogen containing nucleophilic compound such as an amine, for example a diamine, a triamine, an oxyamine, a hydrazine or a hydrazide. In another aspect the scavenger is selected from the group consisting of: diamines, triamines, oxyamines, hydrazines and hydrazides. In another aspect the scavenger is a diamine or a triamine such as ethylenediamine or a derivative thereof, wherein a derivative of ethylenediamine is defined as a compound that is formed from ethylenediamine or that can be imagined to arise from ethylenediamine by replacement of one atom with another atom or group of atoms. In one aspect the derivative of ethylenediamine is a diamine or a triamine which is soluble in a cosolvent. In one aspect the derivative of ethylenediamine is diethylenetriamine. It has thus been found by the inventors that insulin degradation is reduced by the inclusion of ethylenediamine in the non-aqueous lipid pharmaceutical composition according to the invention.

In one aspect the pharmaceutical composition of the invention comprises one or more lipids, one or more surfactants, a scavenger such as ethylenediamine and a cosolvent. In one aspect of the invention the cosolvent is propylene glycol.

In one aspect of the invention, the scavenger is present in combination with the cosolvent.

In one aspect of the invention, the scavenger is present in the pharmaceutical composition in a concentration between from 0.5 mM to 50 mM. In another aspect the scavenger is present in a concentration between from 0.5 mM to 30 mM. In another aspect the scavenger is present in a concentration between from 0.5 mM to 20 mM. In another aspect the scavenger is present in a concentration between from 1 mM to 20 mM. In another aspect the scavenger is present in a concentration between from 1 mM to 10 mM. In another aspect the scavenger is present in a concentration between from 1 mM to 5 mM.

In one aspect of the invention, the insulin is present in the pharmaceutical composition in a concentration between from 0.1 to 30% (w/w) of the total amount of ingredients in the composition. In another aspect the insulin is present in a concentration between from 0.5 to 20% (w/w). In another aspect the insulin is present in a concentration between from 1 to 10% (w/w).

In one aspect of the invention, the insulin is present in the pharmaceutical composition in a concentration between from 0.2 mM to 100 mM. In another aspect the insulin is present in a concentration between from 0.5 to 70 mM. In another aspect the insulin is present in a concentration between from 0.5 to 35 mM. In another aspect the insulin is present in a concentration between from 1 to 30 mM.

In one aspect of the invention, the lipid is present in the pharmaceutical composition in a concentration between from 10% to 90% (w/w) of the total amount of ingredients including insulin in the composition. In another aspect the lipid is present in a concentration between from 10 to 80% (w/w). In another aspect the lipid is present in a concentration between from 10 to 60% (w/w). In another aspect the lipid is present in a concentration between from 15 to 50% (w/w). In another aspect the lipid is present in a concentration between from 15 to 40% (w/w). In another aspect the lipid is present in a concentration between from 20 to 30% (w/w). In another aspect the lipid is present in a concentration of about 25% (w/w).

In one aspect of the invention, the lipid is present in the pharmaceutical composition in a concentration between from 100 mg/g to 900 mg/g of the total amount of ingredients including insulin in the composition. In another aspect the lipid is present in a concentration between from 100 to 800 mg/g. In another aspect the lipid is present in a concentration between from 100 to 600 mg/g. In another aspect the lipid is present in a concentration between from 150 to 500 mg/g. In another aspect the lipid is present in a concentration between from 150 to 400 mg/g. In another aspect the lipid is present in a concentration between from 200 to 300 mg/g. In another aspect the lipid is present in a concentration of about 250 mg/g.

In one aspect of the invention, the cosolvent is present in the pharmaceutical composition in a concentration between from 0% to 30% (w/w) of the total amount of ingredients including insulin in the composition. In another aspect the cosolvent is present in a concentration between from 5% to 30% (w/w). In another aspect the cosolvent is present in a concentration between from 10 to 20% (w/w).

In one aspect of the invention, the cosolvent is present in the pharmaceutical composition in a concentration between from 0 mg/g to 300 mg/g of the total amount of ingredients including insulin in the composition. In another aspect the cosolvent is present in a concentration between from 50 mg/g to 300 mg/g. In another aspect the cosolvent is present in a concentration between from 100 to 200 mg/g.

The term “about” as used herein means in reasonable vicinity of the stated numerical value, such as plus or minus 10%.

The quality of the lipid excipients and/or surfactants as obtained from the manufacturer may also influence the stability of the pharmaceutical composition comprising lipids and/or surfactants. For example certain excipients with higher purity have been identified which stabilize the non-aqueous liquid pharmaceutical composition. It is thus an aspect of the invention that a non-aqueous liquid pharmaceutical composition is obtained wherein the lipid is a high purity lipid. In one aspect a high purity lipid is a lipid which is supplied by the supplier as pharma grade. In one aspect a high purity lipid is a lipid which has an aldehyde and/or ketone content below 20 ppm. In another aspect a high purity lipid is a lipid which has an aldehyde and/or ketone content below 10 ppm. In another aspect a high purity lipid is a lipid which has an aldehyde and/or ketone content below 5 ppm. In another aspect a high purity lipid is a lipid which has an aldehyde and/or ketone content below 2 ppm. In one aspect the lipid is selected from the group consisting of: Glycerol mono-caprylate (such as e.g. Rylo MG08 Pharma) and Glycerol mono-caprate (such as e.g. Rylo MG10 Pharma from Danisco). In another aspect the lipid is selected from the group consisting of: propyleneglycol caprylate (such as e.g. Capmul PG8 from Abitec or Capryol PGMC, or Capryol 90 from Gattefosse).

In one aspect of the invention a non-aqueous liquid pharmaceutical composition comprising at least one surfactant is obtained wherein the surfactant is a high purity surfactant. In one aspect a high purity surfactant is a surfactant which is supplied by the supplier as pharma grade. In one aspect a high purity surfactant is a surfactant which has an aldehyde and/or ketone content below 20 ppm. In another aspect a high purity surfactant is a surfactant which has an aldehyde and/or ketone content below 10 ppm. In another aspect a high purity surfactant is a surfactant which has an aldehyde and/or ketone content below 5 ppm. In another aspect a high purity surfactant is a surfactant which has an aldehyde and/or ketone content below 2 ppm.

The inventors have also found that the stability of the pharmaceutical composition is positively influenced by the use of lipid and/or surfactant excipients which have been purified using a nitrogen containing, surfactant compatible, nucleophilic matrix and/or by purifying the pharmaceutical composition using a nitrogen containing, surfactant compatible, nucleophilic matrix. It has thus been found that nitrogen containing, surfactant compatible, nucleophilic matrix resins, which are normally used in the process of synthesizing small molecule drugs, may be used for removing aldehydes and ketones from lipids, surfactants and/or non-aqueous liquid pharmaceutical compositions according to the invention.

The term “nitrogen containing nucleophilic matrix” or “nitrogen containing nucleophilic resin” is herein used to mean a stationary phase to which compounds may be covalently attached and which comprises an amine such as a diamine or triamine, an oxyamine, a hydrazine or a hydrazide, which is covalently bonded to support particles such as e.g. any kind of organic or inorganic polymeric or oligomeric compound, e.g. polystyrene with different grades of cross linking, polyethylene glycol (PEG), polyethylene glycol attached to polystyrene (e.g. TentaGel), polyacrylamides, polyacrylates, polyurethanes, polycarbonates, polyamides, polysaccharides or silicates or to the inside wall of a column tubing. In one aspect of the invention a nitrogen containing, surfactant compatible, nucleophilic matrix (resin) is a matrix selected from the group consisting of: Hydrazine matrix (resin), hydrazide matrix (resin), oxyamino matrix (resin), diamine matrix (resin) and triamine matrix (resin).

In one aspect the nitrogen containing nucleophilic matrix is compatible with surfactants, herein specified as “surfactant compatible” nucleophilic matrix. The term “surfactant compatible” when used herein in connection with a nitrogen containing nucleophilic matrix refers to a material that can form a homogeneous mixture with a surfactant. In the case of a solid matrix, the term “surfactant compatible” refers to a material that will allow distribution of the surfactant inside the matrix, typically observed as swelling of the matrix (unless the matrix is so extensively cross-linked that it can not swell).

In one aspect of the invention the nitrogen containing nucleophilic matrix is compatible with amphiphilic or hydrophilic surfactants. In one aspect the nitrogen containing nucleophilic matrix is compatible with amphiphilic surfactants. In one aspect the nitrogen containing nucleophilic matrix is compatible with hydrophilic surfactants.

In one aspect the nitrogen containing, surfactant compatible, nucleophilic matrix used according to the invention is selected from the group consisting of: Polymer-bound diethylenetriamine (as e.g. supplied by Aldrich as catalogue no. 494380), polymer-bound ptoluene-sulfonylhydrazide (as e.g. supplied by Aldrich 532339 as catalogue no.) and polymer-bound ethylenediamine (as e.g. supplied by Aldrich as catalogue no. 547484).

In one aspect the nitrogen containing, surfactant compatible, nucleophilic matrix used according to the invention is selected from the group consisting of: p-Toluenesulfonylhydrazide polystyrene matrix, Diethylenetriamine polystyrene matrix, Ethylenediamine matrix stratospheres, Silica tosyl hydrazine matrix, Silica diethylenetriamine matrix, Aminomethacrylate long amine matrix, and Aminomethacrylate short amine matrix.

In one aspect of the invention the purification of the lipid, the cosolvent, the surfactant or the pharmaceutical composition to be purified for aldehydes and/or ketones comprises the steps of:
1) Incubation of the lipid/cosolvent/surfactant/pharmaceutical composition with a nitrogen containing, surfactant compatible, nucleophilic matrix according to the invention, and
2) Isolation such as e.g. filtration, centrifugation or decantation wherein the lipid/cosolvent/surfactant/pharmaceutical composition is isolated from the nitrogen containing, surfactant compatible, nucleophilic matrix.

In one aspect of the invention incubation of the lipid/cosolvent/surfactant/pharmaceutical composition with a nitrogen containing, surfactant compatible, nucleophilic matrix according to the invention is performed at room temperature (r.t.) for 16 hours.

In one aspect of the invention purification of the lipid/cosolvent/surfactant/pharmaceutical composition with a nitrogen containing, surfactant compatible, nucleophilic matrix according to the invention is performed at higher temperature (such as 60° C.) e.g. in order to reduce the viscosity of the pharmaceutical excipients.

In one aspect of the invention the purification of the lipid, the cosolvent, the surfactant or the pharmaceutical composition to be purified for aldehydes and/or ketones is performed by passage through a column comprising a nitrogen containing, surfactant compatible, nucleophilic matrix.

In one aspect of the invention the non-aqueous lipid pharmaceutical composition is stabilized by a combination of two or all of the above mentioned methods, i.e. by combining two or all of the following methods: 1) purification of the lipid and/or pharmaceutical composition on an nitrogen containing, surfactant compatible, nucleophilic matrix, 2) using lipids which are delivered as high purity lipids and 3) adding a scavenger such as ethylene amine to the non-aqueous liquid pharmaceutical composition.

The non-aqueous liquid pharmaceutical composition of the invention may be prepared by conventional techniques, e.g. as described in Remington's Pharmaceutical Sciences, 1985 or in Remington: The Science and Practice of Pharmacy, 19^{th} edition, 1995, where such conventional techniques of the pharmaceutical industry involve dissolving and mixing the ingredients as appropriate to give the desired end product.

In one aspect the method of manufacturing the non-aqueous liquid pharmaceutical composition comprises the step of mixing the ingredients of the composition under inert atmosphere e.g. nitrogen, argon or helium. In one aspect the step of mixing is performed under nitrogen, in another aspect the step of mixing is performed under argon or helium. In one aspect the method of manufacturing the non-aqueous liquid pharmaceutical composition comprises dissolving insulin in the cosolvent in the presence of nitrogen or argon as the first step of the method. In one aspect the method is carried out where absence of oxygen in the reaction mixture is secured in all steps by e.g. carrying out the steps in the presence of nitrogen or argon. In one aspect the method is carried out at 4° C. in all steps. In one aspect the method is carried out at 30° C. in all steps. In one aspect the method is carried out at r.t. in all steps. In one aspect the step of solubilising the insulin is carried out for between 8 to 16 hours. In one aspect the step of mixing the lipid phase with the co-solvent is carried out for about 15 minutes.

The method of manufacturing the non-aqueous liquid pharmaceutical composition may e.g. be carried out in the absence of oxygen, at 4-37° C. and at a pressure of 1-100 bars. In one aspect of the invention the method of manufacturing the composition is carried out at a pressure of 1-20 bars. In one aspect of the invention, where the pharmaceutical composition comprises a cosolvent, said cosolvent is first purified for aldehydes and/or ketones using a nitrogen containing, surfactant compatible, nucleophilic matrix before being added to the composition. In one aspect of the invention a scavenger is dissolved in said purified cosolvent as a first step of the method of manufacturing the pharmaceutical composition, then, as a second step, insulin is dissolved in the scavenger containing cosolvent. In one aspect of the invention the lipid phase consists of one or more different lipids. In one aspect of the invention the lipid phase consists of two or more different lipids. In one aspect of the invention the lipid phase consists of two different lipids. In one aspect the one or more, alternatively two or more, alternatively two lipids are mixed and subsequently purified for aldehydes and ketones using a nitrogen containing, surfactant compatible, nucleophilic matrix before being added to the composition. In one aspect of the invention the lipid phase is mixed with the insulin phase by gentle agitation or stirring.

In one aspect of the invention the method of manufacturing the non-aqueous liquid pharmaceutical composition is carried out in the presence of nitrogen at 22° C. and atmospheric pressure. In one aspect of the invention the cosolvent is purified for aldehyde and ketone impurities on a nitrogen containing, surfactant compatible, nucleophilic matrix before being added to the composition. In one aspect of the invention the purification of the cosolvent comprises: 1) Incubation of the cosolvent such as propylene glycol with a nitrogen containing, surfactant compatible, nucleophilic matrix, followed by 2) an isolation step wherein the cosolvent is isolated. In one aspect the cosolvent such as propylene glycol is mixed with ethylenediamine as a separate step. In one aspect of the invention insulin is dissolved by gentle stirring in a mixture comprising ethylenediamine and propylene glycol. In one aspect a lipid and at least one surfactant are mixed in one step and then purified for aldehydes and/or ketone impurities on a diethylenetriamine matrix in a following step before being added to the non-aqueous liquid pharmaceutical composition. In one aspect a lipid and at least one surfactant are mixed in one step and then purified for aldehydes and/or ketone impurities on a p-toluene-sulfonylhydrazide matrix in a following step before being added to the non-aqueous liquid pharmaceutical composition. In one aspect a lipid and at least one surfactant are mixed in one step and then purified for aldehydes and/or ketone impurities on an ethyleendiamine matrix in a following step before being added to the non-aqueous liquid pharmaceutical composition. In one aspect of the invention a mixture of a lipid and at least one surfactant is mixed by gentle stirring with a mixture comprising an insulin, cosolvent and scavenger such as ethylene diamine.

In one aspect of the invention a method of manufacturing a non-aqueous liquid pharmaceutical composition according to the invention is carried out in the presence of nitrogen at 22° C. and atmospheric pressure by the following consecutive steps:
1) Mixing the cosolvent such as propylene glycol with ethylenediamine
2) Dissolving the insulin by gentle stirring in the mixture of step 1) comprising ethylenediamine and the cosolvent such as propylene glycol
3) Mixing a lipid and at least one surfactant
4) Mixing by gentle stirring the lipid/surfactant mixture of step 3) with the insulin/propylene glycol/ethylenediamine mixture of step 2).

In one aspect of the invention a method of manufacturing a non-aqueous liquid pharmaceutical composition according to the invention is carried out in the presence of nitrogen at 22° C. and atmospheric pressure by the following consecutive steps:
1) Incubating the cosolvent such as propylene glycol with an nitrogen containing, surfactant compatible, nucleophilic matrix,
2) Filtrating the nitrogen containing, surfactant compatible, nucleophilic matrix from the cosolvent such as propylene glycol whereby the cosolvent is isolated
3) Mixing the purified cosolvent such as propylene glycol with ethylenediamine
4) Dissolving the insulin by gentle stirring in the mixture of step 3) comprising ethylenediamine and the purified cosolvent such as propylene glycol
5) Mixing a lipid and at least one surfactant
6) Incubating the mixture of a lipid and at least one surfactant of step 5) with an nitrogen containing, surfactant compatible, nucleophilic matrix such as a diethylenetriamine containing surfactant compatible nucleophilic matrix,
7) Filtrating the nitrogen containing, surfactant compatible, nucleophilic matrix such as a diethylenetriamine containing surfactant compatible nucleophilic matrix from the lipid/surfactant mixture whereby the lipid/surfactant mixture is isolated
8) Mixing by gentle stirring the lipid/surfactant mixture of step 7) with the insulin/propylene glycol/ethylenediamine mixture of step 4).

The terms “water-free” and “non-aqueous” when used for a pharmaceutical composition are used interchangeably herein and refer to a pharmaceutical composition to which no water is added during preparation of the pharmaceutical composition. The insulin and/or one or more of the excipients in the pharmaceutical composition may have small amounts of water bound to it before preparing a pharmaceutical composition according to the invention. In one aspect a water-free pharmaceutical composition according to the invention comprises less than 10% w/w water. In another aspect, the composition according to the invention comprises less than 5% w/w water. In another aspect, the composition according to the invention comprises less than 4% w/w water, in another aspect less than 3% w/w water, in another aspect less than 2% w/w water and in yet another aspect less than 1% w/w water.

The term “stability” is herein used for a non-aqueous liquid pharmaceutical composition to describe the shelf life of the composition. The term “stabilized” or “stable” when referring to a non-aqueous liquid pharmaceutical composition thus refers to a composition with increased physical stability, increased chemical stability or increased physical and chemical stability relative to a non-stabilized or non-stable composition.

The term “physical stability” of the non-aqueous liquid pharmaceutical composition as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the non-aqueous liquid pharmaceutical compositions is evaluated by means of visual inspection and/or turbidity measurements after exposing the composition filled in suitable containers (e.g. cartridges or vials) to mechanical/physical stress (e.g. agitation) at different temperatures for various time periods. Visual inspection of the compositions is performed in a sharp focused light with a dark background. The turbidity of the composition is characterized by a visual score ranking the degree of turbidity for instance on a scale from 0 to 3 (a composition showing no turbidity corresponds to a visual score 0, and a composition showing visual turbidity in daylight corresponds to visual score 3). A composition is classified physical unstable with respect to protein aggregation, when it shows visual turbidity in daylight. Alternatively, the turbidity of the composition can be evaluated by simple turbidity measurements well-known to the skilled person. Physical stability of the non-aqueous liquid pharmaceutical compositions can also be evaluated by using a spectroscopic agent or probe of the conformational status of the protein.

Other small molecules can be used as probes of the changes in protein structure from native to non-native states. For instance “hydrophobic patch” probes that bind preferentially to exposed hydrophobic patches of a protein. These hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the pharmaceutical composition as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the pharmaceutical composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation can be mentioned as another variant of chemical degradation. The chemical stability of the pharmaceutical composition can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, “stabilized” or “stable” when referring to a non-aqueous liquid pharmaceutical composition refers to a non-aqueous liquid pharmaceutical composition with increased physical stability, increased chemical stability or increased physical and chemical stability. In general, a non-aqueous liquid pharmaceutical composition must be stable during use and storage (in compliance with recommended use and storage conditions) until the expiration date is reached.

In one aspect of the invention the non-aqueous liquid pharmaceutical composition comprising the insulin is stable for more than 6 weeks of usage and for more than 2 years of storage.

In another aspect of the invention the non-aqueous liquid pharmaceutical composition comprising the insulin is stable for more than 4 weeks of usage and for more than two years of storage.

In a further aspect of the invention the non-aqueous liquid pharmaceutical composition comprising the insulin is stable for more than 4 weeks of usage and for more than 3 years of storage.

In an even further aspect of the invention the non-aqueous liquid pharmaceutical composition comprising the insulin is stable for more than 2 weeks of usage and for more than two years of storage.

The term “lipid” is herein used for a substance, material or ingredient that is more mixable with oil than with water. A lipid is insoluble or almost insoluble in water but is easily soluble in oil or other nonpolar solvents.

The term “lipid” can comprise one or more lipophilic substances, i.e. substances that form homogeneous mixtures with oils and not with water. Multiple lipids may constitute the lipophilic phase of the non-aqueous liquid pharmaceutical composition and form the oil aspect. At room temperature, the lipid can be solid, semisolid or liquid. For example, a solid lipid can exist as a paste, granular form, powder or flake. If more than one excipient comprises the lipid, the lipid can be a mixture of liquids, solids, or both.

Examples of solid lipids i.e., lipids which are solid or semisolid at room temperature, include, but are not limited to, the following:
1. Mixtures of mono-, di- and triglycerides, such as hydrogenated coco-glycerides (melting point (m.p.) of about 33.5° C. to about 37° C.], commercially-available as WITEPSOL HI5 from Sasol Germany (Witten, Germany); Examples of fatty acid triglycerides e.g., C10-C22 fatty acid triglycerides include natural and hydrogenated oils, such as vegetable oils;
2. Esters, such as propylene glycol (PG) stearate, commercially available as MONOSTEOL (m.p. of about 33° C. to about 36° C.) from Gattefosse Corp. (Paramus, N.J.); diethylene glycol palmito stearate, commercially available as HYDRINE (m.p. of about 44.5° C. to about 48.5° C.) from Gattefosse Corp.;
3. Polyglycosylated saturated glycerides, such as hydrogenated palm/palm kernel oil PEG-6 esters (m.p. of about 30.5° C. to about 38° C.), commercially-available as LABRAFIL M2130 CS from Gattefosse Corp. or Gelucire 33/01;
4. Fatty alcohols, such as myristyl alcohol (m.p. of about 39° C.), commercially available as LANETTE 14 from Cognis Corp. (Cincinnati, Ohio); esters of fatty acids with fatty alcohols, e.g., cetyl palmitate (m.p. of about 50° C.); isosorbid monolaurate, e.g. commercially available under the trade name ARLAMOL ISML from Uniqema (New Castle, Del.), e.g. having a melting point of about 43° C.;
5. PEG-fatty alcohol ether, including polyoxyethylene (2) cetyl ether, e.g. commercially available as BRIJ 52 from Uniqema, having a melting point of about 33° C., or polyoxyethylene (2) stearyl ether, e.g. commercially available as BRIJ 72 from Uniqema having a melting point of about 43° C.;
6. Sorbitan esters, e.g. sorbitan fatty acid esters, e.g. sorbitan monopalmitate or sorbitan monostearate, e.g, commercially available as SPAN 40 or SPAN 60 from Uniqema and having melting points of about 43° C. to 48° C. or about 53° C. to 57° C. and 41° C. to 54° C., respectively; and
7. Glyceryl mono-C6-C14-fatty acid esters. These are obtained by esterifying glycerol with vegetable oil followed by molecular distillation. Monoglycerides include, but are not limited to, both symmetric (i.e. β-monoglycerides) as well as asymmetric monoglycerides (α-monoglycerides). They also include both uniform glycerides (in which the fatty acid constituent is composed primarily of a single fatty acid) as well as mixed glycerides (i.e. in which the fatty acid constituent is composed of various fatty acids). The fatty acid constituent may include both saturated and unsaturated fatty acids having a chain length of from e.g. C8-C14. Particularly suitable are glyceryl mono laurate e.g. commercially available as IMWITOR 312 from Sasol North America (Houston, Tex.), (m.p. of about 56° C.-60° C.); glyceryl mono dicocoate, commercially available as IMWITOR 928 from Sasol (m.p. of about 33° C.-37° C.); monoglyceryl citrate, commercially available as IMWITOR 370, (m.p. of about 59 to about 63° C.); or glyceryl mono stearate, e.g., commercially available as IMWITOR 900 from Sasol (rn.p. of about 56° C.-61° C.); or self-emulsifying glycerol mono stearate, e.g., commercially available as IMWITOR 960 from Sasol (m.p. of about 56° C.-61° C.).

Examples of liquid and semisolid lipids, i.e., lipids which are liquid or semisolid at room temperature include, but are not limited to, the following:
1. Mixtures of mono-, di- and triglycerides, such as medium chain mono- and diglycerides, glyceryl caprylate/caprate, commercially-available as CAPMUL MCM from Abitec Corp. (Columbus, Ohio); and glycerol monocaprylate, commercially available as RYLO MG08 Pharma and glycerol monocaprate, commercially available as RYLO MG10 Pharma from DANISCO.
2. Glyceryl mono- or di fatty acid ester, e.g. of C6-C18, e.g. C6-C16 e.g. C8-C10, e.g. C8, fatty acids, or acetylated derivatives thereof, e.g. MYVACET 9-45 or 9-08 from Eastman Chemicals (Kingsport, Tenn.) or IMWITOR 308 or 312 from Sasol;
3. Propylene glycol mono- or di-fatty acid ester, e.g. of C8-C20, e.g. C8-C12, fatty acids, e.g. LAUROGLYCOL 90, SEFSOL 218, or CAPRYOL 90 or CAPMUL PG-8 (same as propylene glycol caprylate) from Abitec Corp. or Gattefosse;
4. Oils, such as safflower oil, sesame oil, almond oil, peanut oil, palm oil, wheat germ oil, corn oil, castor oil, coconut oil, cotton seed oil, soybean oil, olive oil and mineral oil;
5. Fatty acids or alcohols, e.g. C8-C20, saturated or mono- or di-unsaturated, e.g. oleic acid, oleyl alcohol, linoleic acid, capric acid, caprylic acid, caproic acid, tetradecanol, dodecanol, decanol;
6. Medium chain fatty acid triglycerides, e.g. C8-C12, e.g. MIGLYOL 812, or long chain fatty acid triglycerides, e.g. vegetable oils;
7. Transesterified ethoxylated vegetable oils, e.g. commercially available as LABRAFIL M2125 CS from Gattefosse Corp;
8. Esterified compounds of fatty acid and primary alcohol, e.g. C8-C20, fatty acids and C2-C3 alcohols, e.g. ethyl linoleate, e.g. commercially available as NIKKOL VF-E from Nikko Chemicals (Tokyo, Japan), ethyl butyrate, ethyl caprylate oleic acid, ethyl oleate, isopropyl myristate and ethyl caprylate;
9. Essential oils, or any of a class of volatile oils that give plants their characteristic odours, such as spearmint oil, clove oil, lemon oil and peppermint oil;
10. Fractions or constituents of essential oils, such as menthol, carvacrol and thymol;
11. Synthetic oils, such as triacetin, tributyrin;
12. Triethyl citrate, acetyl triethyl citrate, tributyl citrate, acetyl tributyl citrate;
13. Polyglycerol fatty acid esters, e.g. diglyceryl monooleate, e.g. DGMO-C, DGMO-90, DGDO from Nikko Chemicals; and
14. Sorbitan esters, e.g. sorbitan fatty acid esters, e.g. sorbitan monolaurate, e.g. commercially available as SPAN 20 from Uniqema.
15. Phospholipids, e.g. Alkyl-O-Phospholipids, Diacyl Phosphatidic Acids, Diacyl Phosphatidyl Cholines, Diacyl Phosphatidyl Ethanolamines, Diacyl Phosphatidyl Glycerols, Di-O-Alkyl Phosphatidic Acids, L-alpha-Lysophosphatidylcholines (LPC), Lalpha-Lysophosphatidylethanolamines (LPE), L-alpha-Lysophosphatidylglycerol (LPG), Lalpha-Lysophosphatidylinositols (LPI), L-alpha-Phosphatidic acids (PA), L-alphaPhosphatidylcholines (PC), L-alpha-Phosphatidylethanolamines (PE), L-alphaPhosphatidylglycerols (PG), Cardiolipin (CL), L-alpha-Phosphatidylinositols (PI), L-alphaPhosphatidylserines (PS), Lyso-Phosphatidylcholines, Lyso-Phosphatidylglycerols, snGlycerophosphorylcholines commercially available from LARODAN, or soybean phospholipid (Lipoid S100) commercially available from Lipoid GmbH.
16. Polyglycerol fatty acid esters, such as polyglycerol oleate (Plurol Oleique from Gattefosse).

In one aspect of the invention, the lipid is one or more selected from the group consisting of mono-, di-, and triglycerides. In a further aspect, the lipid is one or more selected from the group consisting of mono- and diglycerides. In yet a further aspect, the lipid is Capmul MCM or Capmul PG-8. In a still further aspect, the lipid is Capmul PG-8. In a further aspect the lipid is Glycerol monocaprylate (Rylo MG08 Pharma from Danisco).

In one aspect the cosolvent according to the invention is a cosolvent which is a semi-polar protic cosolvent and refers to a hydrophilic, water miscible carbon-containing cosolvent that contains one or more alcohol or amine functional groups or mixtures thereof. The polarity is reflected in the dielectric constant or the dipole moment of a solvent. The polarity of a solvent determines what type of compounds it is able to dissolve and with what other solvents or liquid compounds it is miscible. Typically, polar solvents dissolve polar compounds best and non-polar cosolvents dissolve non-polar compounds best: “like dissolves like”. Strongly polar compounds such as inorganic salts (e.g. sodium chloride) dissolve only in very polar solvents.

Semi-polar cosolvents are here defined as cosolvents with a dielectric constant in the range of 20-50, whereas polar and non-polar cosolvents are defined by a dielectric constant above 50 and below 20, respectively. Examples of semi-polar protic are listed in Table 1 together with water as a reference.

TABLE 1
Dielectric constants (static permittivity) of selected semi-
polar organic protic cosolvents and water as a reference
(Handbook of Chemistry and Physics, CMC Press, dielectricity
constants are measured in static electric fields or at relatively
low frequencies, where no relaxation occurs).

| Solvent (Temperature, Kelvin) | Dielectric constant, ∈* |
|---|---|
| Water (293.2) | 80.1 |
| Propanetriol [Glycerol] (293.2) | 46.53 |
| Ethanediol [Ethylene Glycol] (293.2) | 41.4 |
| 1,3-propanediol (293.2) | 35.1 |
| Methanol (293.2) | 33.0 |
| 1,4-butanediol (293.2) | 31.9 |
| 1,3-butanediol (293.2) | 28.8 |
| 1,2-propanediol [propylene | 27.5 |
| glycol] (303.2) | |
| Ethanol (293.2) | 25.3 |
| Isopropanol (293.2) | 20.18 |

In the present context, 1,2-propanediol and propylene glycol are used interchangeable. In the present context, propanetriol and glycerol are used interchangeably. In the present context, ethanediol and ethylene glycol are used interchangeably.

In one aspect of the invention, the cosolvent is selected from the group consisting of polyols. The term “polyol” as used herein refers to chemical compounds containing multiple hydroxyl groups.

In a further aspect of the invention, the cosolvent is selected from the group consisting of diols and triols. The term “diol” as used herein refers to chemical compounds containing two hydroxyl groups. The term “triol” as used herein refers to chemical compounds containing three hydroxyl groups.

In a further aspect of the invention, the cosolvent is selected from the group consisting of glycerol (propanetriol), ethanediol (ethylene glycol), 1,3-propanediol, methanol, 1,4-butanediol, 1,3-butanediol, propylene glycol (1,2-propanediol), ethanol and isopropanol, or mixtures thereof. In a further aspect of the invention, the cosolvent is selected from the group consisting of propylene glycol and glycerol. In a preferred aspect of the invention, the cosolvent is glycerol. This cosolvent is biocompatible even at high dosages and has a high cosolvent capacity for insulin peptides compounds. In another preferred aspect of the invention, the cosolvent is selected from the group consisting of propylene glycol and ethylene glycol. These cosolvents have a low viscosity, are biocompatible at moderate doses, and have very high cosolvent capacity for insulin peptides. In a further aspect of the invention, the cosolvent is propylene glycol.

In one aspect, the cosolvent of the formulation is propylene glycol USP/EP with a purity of at least 99.8% (such as Propylene glycol USP/EP from Dow Chemical).

In one aspect of the invention, the cosolvent has an aldehyde content below 5 ppm.

In another aspect of the invention, the cosolvent has an aldehyde content below 2 ppm.

In one aspect, the cosolvent is propylene glycol which has an aldehyde content below 2 ppm.

Aldehyde content in semipolar organic solvents such as propylene glycol can be analysed with the method described in example 9.

In one aspect the aqueous pharmaceutical composition according to the invention comprises one or more surfactants, such as a mixture of surfactants, or surface active agents, which reduce interfacial tension. The surfactant is e.g., nonionic, ionic or amphoteric. Surfactants can be complex mixtures containing side products or un-reacted starting products involved in the preparation thereof, e.g., surfactants made by polyoxyethylation may contain another side product, e.g., PEG. The surfactant or surfactants according to the invention have a hydrophilic-lipophilic balance (HLB) value which is at least 8. For example, the surfactant may have a mean HLB value of 8-30, e.g., 12-30, 12-20 or 13-15. The surfactants can be liquid, semisolid or solid in nature.

The Hydrophilic-lipophilic balance (HLB) of a surfactant is a measure of the degree to which it is hydrophilic or lipophilic, determined by calculating values for the different regions of the molecule, as described by Griffin (Griffin WC: “Classification of Surface-Active Agents by ‘FILB,’” Journal of the Society of Cosmetic Chemists 1 (1949): 311) or by Davies (Davies JT: “A quantitative kinetic theory of emulsion type, I. Physical chemistry of the emulsifying agent,” Gas/Liquid and Liquid/Liquid Interface. Proceedings of the International Congress of Surface Activity (1957): 426-438).

The term “surfactant” as used herein refers to any substance, in particular a detergent that can adsorb at surfaces and interfaces, e.g. liquid to air, liquid to liquid, liquid to container or liquid to any solid. The surfactant may be selected from a detergent, such as ethoxylated castor oil, polyglycolyzed glycerides, acetylated monoglycerides, sorbitan fatty acid esters, polysorbate, such as polysorbate-20, poloxamers, such as poloxamer 188 and poloxamer 407, polyoxyethylene sorbitan fatty acid esters, polyoxyethylene derivatives such as alkylated and alkoxylated derivatives (tweens, e.g. Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, cephalins, phosphatidyl serine), glyceroglycolipids (galactopyransoide), sphingophospholipids (sphingomyelin), and sphingoglycolipids (ceramides, gangliosides), DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), alkyl, alkoxyl (alkyl ester), alkoxy (alkyl ether)- derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the postively charged DODAC, DOTMA, DCP, BISHOP, lysophosphatidylserine and lysophosphatidylthreonine, zwitterionic surfactants (e.g. N-alkyl-N,N-dimethylammonio-1-propanesulfonates, 3-cholamido-1-propyldimethylammonio-1-propanesulfonate, dodecylphosphocholine, myristoyl lysophosphatidylcholine, hen egg lysolecithin), cationic surfactants (quaternary ammonium bases) (e.g. cetyltrimethylammonium bromide, cetylpyridinium chloride), non-ionic surfactants (e.g. alkyl glucosides like dodecyl β-D-glucopyranoside, dodecyl β-D-maltoside, tetradecyl β-D-glucopyranoside, decyl β-D-maltoside, dodecyl β-D-maltoside, tetradecyl β-D-maltoside, hexadecyl β-D-maltoside, decyl β-D-maltotrioside, dodecyl β-D-maltotrioside, tetradecyl β-D-maltotrioside, hexadecyl β-D-maltotrioside, n-dodecyl-sucrose, n-decyl-sucrose, fatty alcohol ethoxylates (e.g. polyoxyethylene alkyl ethers like octaethylene glycol mono tridecyl ether, octaethylene glycol mono dodecyl ether, octaethylene glycol mono tetradecyl ether), block copolymers as polyethyleneoxide/polypropyleneoxide block copolymers (Pluronics/Tetronics, Triton X-100) ethoxylated sorbitan alkanoates surfactants (e.g. Tween-20, Tween-40, Tween-80, Brij-35), fusidic acid derivatives (e.g. sodium tauro-dihydrofusidate etc.), longchain fatty acids and salts thereof C8-C20 (eg. oleic acid and caprylic acid), acylcarnitines and derivatives, N-acylated derivatives of lysine, arginine or histidine, or side-chain acylated derivatives of lysine or arginine, N-acylated derivatives of dipeptides comprising any combination of lysine, arginine or histidine and a neutral or acidic amino acid, N-acylated derivative of a tripeptide comprising any combination of a neutral amino acid and two charged amino acids, or the surfactant may be selected from the group of imidazoline derivatives, or mixtures thereof.

Examples of solid surfactants include, but are not limited to:
1. Reaction products of a natural or hydrogenated castor oil and ethylene oxide. The natural or hydrogenated castor oil may be reacted with ethylene oxide in a molar ratio from about 1:35 to about 1:60, with optional removal of the PEG component from the products. Various such surfactants are commercially available, e-g., the CREMOPHOR series from BASF Corp. (Mt. Olive, N.J.), for example CREMOPHOR RH 40 which is PEG40 hydrogenated castor oil which has a saponification value of about 50- to 60, an acid value less than about one, a water content, i.e., Fischer, less than about 2%, an n_{D}⁶⁰ of about 1.453-1.457, and an HLB of about 14-16;
2. Polyoxyethylene fatty acid esters that include polyoxyethylene stearic acid esters, such as the MYRJ series from Uniqema e.g., MYRJ 53 having an m.p. of about 47° C.
Particular compounds in the MYRJ series are, e.g., MYRJ 53 having an m.p. of about 47° C. and PEG-40-stearate available as MYRJ 52;
3. Sorbitan derivatives that include the TWEEN series from Uniqema, e.g., TWEEN 60;
4. Polyoxyethylene-polyoxypropylene co-polymers and block co-polymers or poloxamers, e.g., Pluronic F127, Pluronic F68 from BASF;
5. Polyoxyethylene alkyl ethers, e.g., polyoxyethylene glycol ethers of C₁₂-C₁₈ alcohols, polyoxyl 10- or 20-cetyl ether or polyoxyl 23-lauryl ether, or 20-oleyl ether, or polyoxyl 10-, 20- or 100-stearyl ether, known and commercially available as the BRIJ series from Uniqema. Particularly useful products from the BRIJ series are BRIJ 58; BRIJ 76; BRIJ 78; BRIJ 35, i.e. polyoxyl 23 lauryl ether; and BRIJ 98, i.e. polyoxyl 20 oleyl ether. These products have an m.p. between about 32° C. to about 43° C.;
6. Water-soluble tocopheryl PEG succinic acid esters available from Eastman Chemical Co. with an m.p. of about 36° C., e.g, TPGS and vitamin E TPGS.
7. PEG sterol ethers having, e.g., from 5-35 [CH₂—CH, —O] units, e.g., 20-30 units, e-g., SOLULAN C24 (Choleth-24 and Cetheth-24) from Chemron (Paso Robles, Calif.); similar products which may also be used are those which are known and commercially available as NIKKOL BPS-30 (polyethoxylated 30 phytosterol) and NIKKOL BPSH-25 (polyethoxylated 25 phytostanol) from Nikko Chemicals;
8. Polyglycerol fatty acid esters, e.g., having a range of glycerol units from 4-10, or 4, 6 or 10 glycerol units. For example, particularly suitable are deca-/hexa-/tetraglyceryl monostearate, e.g., DECAGLYN, HEXAGLYN and TETRAGLYN from Nikko Chemicals;
9. Alkylene polyol ether or ester, e.g., lauroyl macrogol-32 glycerides and/or stearoyl macrogol-32 glycerides which are GELUCIRE 44/14 and GELUCIRE 50/13 respectively;
10. Polyoxyethylene mono esters of a saturated C₁₀ to C₂₂, such as C₁₈ substituted e.g. hydroxy fatty acid; e.g. 12 hydroxy stearic acid PEG ester, e.g. of PEG about e.g. 600-900 e.g. 660 Daltons MW, e.g. SOLUTOL HS 15 from BASF (Ludwigshafen, 20 Germany). According to a BASF technical leaflet MEF 151E (1986), SOLUTOL HS 15 comprises about 70% polyethoxylated 12-hydroxystearate by weight and about 30% by weight unesterified polyethylene glycol component. It has a hydrogenation value of 90 to 110, a saponification value of 53 to 63, an acid number of maximum 1, and a maximum water content of 0.5% by weight;
11. Polyoxyethylene-polyoxypropylene-alkyl ethers, e.g. polyoxyethylene-polyoxypropyleneethers of C₁₂ to C₁₈ alcohols, e.g. polyoxyethylen-20-polyoxypropylene-4-cetylether which is commercially available as NIKKOL PBC 34 from Nikko Chemicals;
12. Polyethoxylated distearates, e.g. commercially available under the tradenames ATLAS G 1821 from Uniqema and NIKKOCDS-6000P from Nikko Chemicals; and
13. Lecithins, e.g. soy bean phospholipid, e.g. commercially available as LIPOID S75 from Lipoid GmbH (Ludwigshafen, Germany) or egg phospholipid, commercially available as PHOSPHOLIPON 90 from Nattermann Phospholipid (Cologne, Germany).

Examples of liquid surfactants include, but are not limited to, sorbitan derivatives such as TWEEN 20, TWEEN 40 and TWEEN 80, SYNPERONIC L44, and polyoxyl 10-oleyl ether, all available from Uniqema, and polyoxyethylene containing surfactants e.g. PEG-8 caprylic/capric glycerides (e.g. Labrasol or Labrasol ALF available from Gattefosse).

The composition of the invention may comprise from about 0% to about 95% by weight surfactant, e.g. from about 5% to about 80% by weight, e.g., about 10% to about 70% by weight, e.g. from about 20% to about 60% by weight, e.g. from about 30% to about 50%.

In one aspect of the invention, the surfactant is polyoxyethylene-polyoxypropylene co-polymers and block co-polymers or poloxamers, e.g., Pluronic F127, Pluronic F68 from BASF.

In one aspect of the invention, the surfactant is a poloxamer. In a further aspect, the surfactant is selected from the group consisting of poloxamer 188, poloxamer 407 and mixtures of poloxamer 407 and poloxamer 188.

In one aspect of the invention, the surfactant is a polyoxyethylene containing surfactants e.g. PEG-8 caprylic/capric glycerides (e.g. Labrasol available from Gattefosse).

In a further aspect of the invention, the surfactant is a polyoxyethylene containing surfactants e.g. PEG-8 caprylic/capric glycerides which has an aldehyde content below 10 ppm. (Labrasol ALF available from Gattefosse).

In yet a further aspect of the invention, the surfactant is a polyoxyethylene containing surfactants e.g. PEG-8 caprylic/capric glycerides which has an aldehyde content below 5 ppm. (Labrasol ALF available from Gattefosse).

The aldehyde content in PEG-8 caprylic/capric glycerides (Labrasol) can e.g. be analysed by NMR (see example 14).

In one aspect of the invention, the surfactant is polyethylene glycol sorbitan monolaurate (e.g. Tween 20 available from Merck or Croda).

In one aspect of the invention, the surfactant is super refined polysorbate 20 (e.g. Tween 20 available from Croda).

In one aspect of the invention, the surfactant is super refined polysorbate 20 (e.g. Tween 20 available from Croda) which has an aldehyde content below 10 ppm.

In a further aspect of the invention, the surfactant is super refined polysorbate 20 (e.g. Tween 20 available from Croda) which has an aldehyde content below 5 ppm.

In yet a further aspect of the invention, the surfactant is super refined polysorbate 20 (e.g. Tween 20 available from Croda) which has an formaldehyde content below 3 ppm.

In one aspect of the invention, the surfactant is polyoxyethylene sorbitan monooleate (e.g. Tween 80 available from Merck or Croda).

In one aspect of the invention, the surfactant is super refined polysorbate 80 (e.g. Tween 80 available from Croda).

In a further aspect of the invention, the surfactant is super refined polysorbate 80 (e.g. Tween 80 available from Croda) which has an aldehyde content below 10 ppm.

In yet a further aspect of the invention, the surfactant is super refined polysorbate 80 (e.g. Tween 80 available from Croda) which has an aldehyde content below 5 ppm.

In yet a further aspect of the invention, the surfactant is super refined polysorbate 80 (e.g. Tween 80 available from Croda) which has a formaldehyde content below 3 ppm.

In one aspect of the invention, the surfactant is Cremophor RH40 from BASF.

In one aspect of the invention, the surfactant is polyglycerol-2-caprylate or polyglycerol-2-caprate.

In certain aspects of the present invention, the non-aqueous liquid pharmaceutical composition may comprise one or more additional excipients commonly found in pharmaceutical compositions. Examples of such excipients include, but are not limited to, antioxidants, antimicrobial agents, enzyme inhibitors, stabilizers, preservatives, flavors, sweeteners and other components as described in Handbook of Pharmaceutical Excipients, Rowe et al., Eds., 4'h Edition, Pharmaceutical Press (2003), which is hereby incorporated by reference.

These additional excipients may be in a concentration from about 0.05-5% by weight of the total pharmaceutical composition. Antioxidants, anti-microbial agents, enzyme inhibitors, stabilizers or preservatives typically make up to about 0.05-1% by weight of the total pharmaceutical composition. Sweetening or flavoring agents typically make up to about 2.5% or 5% by weight of the total pharmaceutical composition.

Examples of antioxidants include, but are not limited to, ascorbic acid and its derivatives, tocopherol and its derivatives, butyl hydroxyl anisole and butyl hydroxyl toluene.

In one aspect the one or more additional excipients are one or more selected from the group consisting of: Amino acids and di-amino acids like phe-phe or arg-arg.

With “insulin”, “an insulin” or “the insulin” as used herein is meant human insulin, porcine insulin or bovine insulin with disulfide bridges between CysA7 and CysB7 and between CysA20 and CysB19 and an internal disulfide bridge between CysA6 and CysA11 or an insulin analogue or derivative thereof.

Human insulin consists of two polypeptide chains, the A and B chains which contain 21 and 30 amino acid residues, respectively. The A and B chains are interconnected by two disulphide bridges. Insulin from most other species is similar, but may contain amino acid substitutions in some positions.

An insulin analogue as used herein is a polypeptide which has a molecular structure which formally can be derived from the structure of a naturally occurring insulin, for example that of human insulin, by deleting and/or substituting at least one amino acid residue occurring in the natural insulin and/or by adding at least one amino acid residue.

In one aspect an insulin analogue according to the invention comprises less than 8 modifications (substitutions, deletions, additions) relative to human insulin. In one aspect an insulin analogue comprises less than 7 modifications (substitutions, deletions, additions) relative to human insulin. In one aspect an insulin analogue comprises less than 6 modifications (substitutions, deletions, additions) relative to human insulin. In another aspect an insulin analogue comprises less than 5 modifications (substitutions, deletions, additions) relative to human insulin. In another aspect an insulin analogue comprises less than 4 modifications (substitutions, deletions, additions) relative to human insulin. In another aspect an insulin analogue comprises less than 3 modifications (substitutions, deletions, additions) relative to human insulin. In another aspect an insulin analogue comprises less than 2 modifications (substitutions, deletions, additions) relative to human insulin.

A derivative of insulin according to the invention is a naturally occurring human insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin.

A derivative of insulin is thus human insulin or an insulin analogue which comprises at least one covalent modification such as a side-chain attached to one or more amino acids of the insulin peptide.

Herein, the naming of the insulin is done according to the following principles: The names are given as mutations and modifications (acylations) relative to human insulin. With “desB30 human insulin” is thus meant an analogue of human insulin lacking the B30 amino acid residue. Similarly, “desB29desB30 human insulin” means an analogue of human insulin lacking the B29 and B30 amino acid residues. With “B1”, “Al” etc. is meant the amino acid residue at position 1 in the B-chain of insulin (counted from the N-terminal end) and the amino acid residue at position 1 in the A-chain of insulin (counted from the N-terminal end), respectively. The amino acid residue in a specific position may also be denoted as e.g. PheB1 which means that the amino acid residue at position B1 is a phenylalanine residue.

For the naming of the acyl moiety, the naming is done according to IUPAC nomenclature and in other cases as peptide nomenclature. For example, naming the acyl moiety:

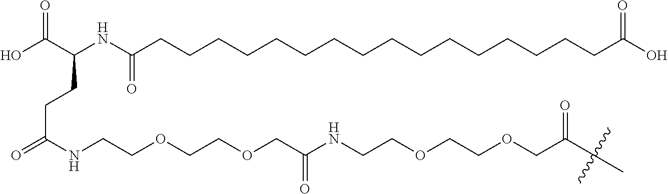 [SMILES: *C(=O)COCCOCCNC(=O)COCCOCCNC(=O)CC[C@H](NC(=O)CCCCCCCCCCCCCCCCC(=O)O)C(=O)O]

can be e.g. “octadecanedioyl-γ-L-Glu-OEG-OEG”, or “17-carboxyheptadecanoyl-γ-L-GluOEG-OEG”, wherein OEG is short hand notation for the amino acid —NH(CH₂)₂—O—(CH₂)₂OCH₂CO—, and γ-L-Glu (or g-L-Glu) is short hand notation for the L-form of the amino acid gamma glutamic acid moiety.

The acyl moiety of the modified peptides or proteins may be in the form of a pure enantiomer wherein the stereo configuration of the chiral amino acid moiety is either D or L (or if using the R/S terminology: either R or S) or it may be in the form of a mixture of enantiomers (D and L/R and S). In one aspect of the invention the acyl moiety is in the form of a mixture of enantiomers. In one aspect the acyl moiety is in the form of a pure enantiomer. In one aspect the chiral amino acid moiety of the acyl moiety is in the L form. In one aspect the chiral amino acid moiety of the acyl moiety is in the D form.

In one aspect a derivative of insulin in a non-aqueous liquid pharmaceutical composition according to the invention is an insulin peptide that is acylated in one or more amino acids of the insulin peptide.

In one aspect a derivative of insulin in a non-aqueous liquid pharmaceutical composition according to the invention is an insulin peptide that is stabilised towards proteolytic degradation (by specific mutations) and further acylated at the B29-lysine. A non-limiting example of insulin peptides that are stabilised towards proteolytic degradation (by specific mutations) may e.g. be found in WO 2008/034881, which is hereby incorporated by reference.

A non-limiting example of acylated polypeptides may e.g. be found in the patent application WO 2009/115469 (PCT application number PCT/EP2009/053017) such as acylated polypeptides as described in the passage beginning on page 25, line 3 (page 24 of PCT/EP2009/053017).

In one aspect of the invention, the derivative of insulin in a non-aqueous liquid pharmaceutical composition according to the invention is an acylated insulin which is found in WO 2009/115469 (PCT application number PCT/EP2009/053017), such as the acylated insulins listed in claim 8 in WO 2009/115469.

In one aspect of the invention, the derivative of insulin is selected from the group consisting of:

B29K(N(∈)hexadecanedioyl-γ-L-Glu) A14E B25H desB30 human insulin
B29K(N(∈)octadecanedioyl-γ-L-Glu-OEG-OEG) desB30 human insulin
B29K(N(∈)octadecanedioyl-γ-L-Glu) A14E B25H desB30 human insulin
B29K(N(∈)eicosanedioyl-γ-L-Glu) A14E B25H desB30 human insulin
B29K(N(∈)octadecanedioyl-γ-L-Glu-OEG-OEG) A14E B25H desB30 human insulin
B29K(N(∈)eicosanedioyl-γ-L-Glu-OEG-OEG) A14E B25H desB30 human insulin
B29K(N(∈)eicosanedioyl-γ-L-Glu-OEG-OEG) A14E B16H B25H desB30 human insulin
B29K(N(∈)hexadecanedioyl-γ-L-Glu) A14E B16H B25H desB30 human insulin
B29K(N(∈)eicosanedioyl-γ-L-Glu-OEG-OEG) A14E B16H B25H desB30 human insulin
B29K(N(∈)octadecanedioyl) A14E B25H desB30 human insulin.

In another aspect of the invention, the derivative of insulin is B29K(N(∈)octadecanedioyl-γ-L-Glu-OEG-OEG) A14E B25H desB30 human insulin.

The following is a non-limiting list of aspects according to the invention:

1. A non-aqueous liquid pharmaceutical composition comprising at least one lipid, at least one insulin, at least one scavenger and optionally at least one surfactant, wherein the scavenger is a nitrogen containing nucleophilic compound
2. A non-aqueous liquid pharmaceutical composition according to aspect 1, wherein the scavenger is an amine.
3. A non-aqueous liquid pharmaceutical composition according to aspect 1 or 2, wherein the scavenger is selected from the group consisting of a diamine, a triamine, an oxyamine, a hydrazine and a hydrazide.
4. A non-aqueous liquid pharmaceutical composition according to aspect 3, wherein the scavenger is a diamine.
5. A non-aqueous liquid pharmaceutical composition according to anyone of the preceding aspects, wherein the scavenger is present in the composition in a concentration from between 0.1 mM to 5.0 mM.
6. A non-aqueous liquid pharmaceutical composition according to anyone of the preceding aspects, wherein the scavenger is present in the composition in a concentration from between 0.1 mM to 3.0 mM.
7. A non-aqueous liquid pharmaceutical composition according to anyone of the preceding aspects, wherein the scavenger is present in the composition in a concentration from between 0.1 mM to 1.0 mM.
8. A non-aqueous liquid pharmaceutical composition according to anyone of the preceding aspects, wherein the scavenger is present in the composition in a concentration from between 0.2 mM to 0.8 mM.
9. A non-aqueous liquid pharmaceutical composition according to aspect 7, wherein the scavenger is present in the composition in a concentration from between 0.1 mM to 0.5 mM.
10. A non-aqueous liquid pharmaceutical composition according to aspect 7, wherein the scavenger is present in the composition in a concentration from between 0.5 mM to 1.0 mM.
11. A non-aqueous liquid pharmaceutical composition according to anyone of the preceding aspects, wherein the scavenger is ethylenediamine or a derivative thereof.
12. A non-aqueous liquid pharmaceutical composition according to anyone of the preceding aspects, wherein the scavenger is ethylenediamine.
13. A non-aqueous liquid pharmaceutical composition according to anyone of the preceding aspects, wherein the lipid and/or surfactant is a high purity lipid.
14. A non-aqueous liquid pharmaceutical composition according to anyone of the preceding aspects, wherein the lipid and/or surfactant is pharma grade.
15. A non-aqueous liquid pharmaceutical composition according to anyone of the preceding aspects, wherein the lipid and/or surfactant has an aldehyde and/or ketone content below 20 ppm when added to the pharmaceutical composition.
16. A non-aqueous liquid pharmaceutical composition according aspect 15, wherein the lipid and/or surfactant has an aldehyde and/or ketone content below 10 ppm.
17. A non-aqueous liquid pharmaceutical composition according aspect 16, wherein the lipid and/or surfactant has an aldehyde and/or ketone content below 5 ppm.
18. A non-aqueous liquid pharmaceutical composition according aspect 17, wherein the lipid and/or surfactant has an aldehyde and/or ketone content below 2 ppm.
19. A non-aqueous liquid pharmaceutical composition according to anyone of the preceding aspects, wherein the lipid and/or surfactant has been purified using a nitrogen containing oil compatible nucleophilic matrix before being added to the pharmaceutical composition.
20. A non-aqueous liquid pharmaceutical composition according to anyone of the preceding aspects, wherein the lipid and/or surfactant is selected from the group consisting of: Glycerol mono-caprylate (such as e.g. Rylo MG08 Pharma), Glycerol mono-caprate (such as e.g. Rylo MG10 Pharma from Danisco), polyglycerol fatty acid ester (such as e.g. Plurol Oleique or Diglycerol monocaprylate), caprylocaproyl macrogol-8-glycerides (such as e.g. Labrasol ALF), polysorbate 20 (such as Tween 20 or super refined Tween 20) and polysorbate 80 (such as Tween 80 or super refined Tween 80).
21. A non-aqueous liquid pharmaceutical composition according to anyone of the preceding aspects, wherein the lipid and/or surfactant is selected from the group consisting of: Glycerol mono-caprylate (such as e.g. Rylo MG08 Pharma) and Glycerol mono-caprate (such as e.g. Rylo MG10 Pharma from Danisco).
22. A non-aqueous liquid pharmaceutical composition according to anyone of the preceding aspects further comprising a cosolvent.
23. A non-aqueous liquid pharmaceutical composition according to aspect 22 wherein the cosolvent is propylene glycol.
24. A non-aqueous liquid pharmaceutical composition according to anyone of the preceding aspects further comprising a surfactant.
25. A non-aqueous liquid pharmaceutical composition according to aspect 24 wherein the surfactant is a non-ionic surfactant.
26. A non-aqueous liquid pharmaceutical composition according to aspect 25 wherein the non-ionic surfactant is selected from the group consisting of: Ethoxylated sorbitan alkanoates surfactants and PEG-8 caprylic/capric glycerides.
27. A non-aqueous liquid pharmaceutical composition according to anyone of the preceding aspects, wherein the insulin is human insulin, a human insulin analogue or a derivative of one of these.
28. A non-aqueous liquid pharmaceutical composition according to aspect 27, wherein the insulin is a derivative of insulin.
29. A non-aqueous liquid pharmaceutical composition according to aspect 27, wherein the insulin is a derivative of insulin which is selected from the group consisting of:
B29K(N(∈)hexadecanedioyl-γ-L-Glu) A14E B25H desB30 human insulin
B29K(N(∈)octadecanedioyl-γ-L-Glu-OEG-OEG) desB30 human insulin
B29K(N(∈)octadecanedioyl-γ-L-Glu) A14E B25H desB30 human insulin
B29K(N(∈)eicosanedioyl-γ-L-Glu) A14E B25H desB30 human insulin
B29K(N(∈)octadecanedioyl-γ-L-Glu-OEG-OEG) A14E B25H desB30 human insulin
B29K(N(∈)eicosanedioyl-γ-L-Glu-OEG-OEG) A14E B25H desB30 human insulin
B29K(N(∈)eicosanedioyl-γ-L-Glu-OEG-OEG) A14E B16H B25H desB30 human insulin
B29K(N(∈)hexadecanedioyl-γ-L-Glu) A14E B16H B25H desB30 human insulin
B29K(N(∈)eicosanedioyl-γ-L-Glu-OEG-OEG) A14E B16H B25H desB30 human insulin
B29K(N(∈)octadecanedioyl) A14E B25H desB30 human insulin.
30. A method for manufacturing a non-aqueous liquid pharmaceutical composition according to anyone of the preceding aspects.
31. A method for manufacturing a non-aqueous liquid pharmaceutical composition comprising at least one lipid, at least one insulin, and a cosolvent, wherein said cosolvent, said lipid and said optional surfactant are first purified on a nitrogen containing, surfactant compatible, nucleophilic matrix before being added to the composition.
32. A method for manufacturing a non-aqueous liquid pharmaceutical composition according to aspect 31, wherein the insulin is dissolved in the cosolvent optionally in the presence of nitrogen or argon as a first step.
33. A method for manufacturing a non-aqueous liquid pharmaceutical composition according to anyone of aspect 30-32 comprising the step of mixing the ingredients of the composition under inert atmosphere e.g. nitrogen, argon or helium.
34. A method for manufacturing a non-aqueous liquid pharmaceutical composition according to anyone of aspects 30-33, wherein the reaction is carried out at 4° C. in all steps.
35. A method for manufacturing a non-aqueous liquid pharmaceutical composition according to anyone of aspects 30-34, wherein the reaction is carried out at 30° C. in all steps.
36. A method for manufacturing a non-aqueous liquid pharmaceutical composition according to anyone of aspects 30-35, wherein the reaction is carried out at room temperature (r.t.) in all steps.
37. A method for manufacturing a non-aqueous liquid pharmaceutical composition according to anyone of aspects 30-36, wherein the reaction is carried out in the absence of oxygen, at 4-37° C. and at a pressure of 1-100 bars.
38. A method for manufacturing a non-aqueous liquid pharmaceutical composition according to aspect 37, wherein the reaction is carried out at a pressure of 1-20 bars.
39. A method for manufacturing a non-aqueous liquid pharmaceutical composition according to anyone of aspects 30-38, wherein the pharmaceutical composition comprises a cosolvent and a scavenger, wherein the scavenger is dissolved in said purified cosolvent as a first step of the method of manufacturing the pharmaceutical composition, then, as a second step, insulin is dissolved in the scavenger containing cosolvent.
40. A method for manufacturing a non-aqueous liquid pharmaceutical composition according to claim 39, wherein the scavenger is neutralized before being dissolved in said cosolvent.
41. A method for manufacturing a non-aqueous liquid pharmaceutical composition according to claim 40, wherein the scavenger is neutralized by pH adjustment to 6-8.
42. A method for manufacturing a non-aqueous liquid pharmaceutical composition according to claim 40 or 41, wherein the scavenger is dried after neutralization and before being dissolved in said cosolvent.
43. A method for manufacturing a non-aqueous liquid pharmaceutical composition according to claim 42, wherein the scavenger is dried by freeze-drying or spray-drying.
44. A method for manufacturing a non-aqueous liquid pharmaceutical composition according to anyone of aspects 30-43, wherein the lipid phase consists of two or more different lipids.
45. A method for manufacturing a non-aqueous liquid pharmaceutical composition according to anyone of aspects 30-44, wherein the lipid phase is mixed with the insulin phase by gentle agitation or stirring.
46. A method for manufacturing a non-aqueous liquid pharmaceutical composition according to anyone of aspects 30-45, wherein the reaction is carried out in the presence of nitrogen at 22° C. at atmospheric pressure.
47. A method for manufacturing a non-aqueous liquid pharmaceutical composition according to anyone of aspects 30-46, wherein the cosolvent is purified on a nitrogen containing, surfactant compatible, nucleophilic matrix before being added to the composition.
48. A method for manufacturing a non-aqueous liquid pharmaceutical composition according to anyone of aspects 30-47, wherein the insulin is dissolved by gentle stirring in a mixture comprising ethylenediamine and propylene glycol.
49. A method for purifying a lipid, a cosolvent, a surfactant or a pharmaceutical composition comprising a lipid, wherein purification is performed on a nitrogen containing, surfactant compatible, nucleophilic matrix whereby removal of excess aldehyde is achieved.
50. A method for purifying a lipid, a cosolvent, a surfactant or a pharmaceutical composition comprising a lipid according to aspect 49, wherein the nitrogen containing, surfactant compatible, nucleophilic matrix is selected from the group consisting of: A hydrazine matrix, a hydrazide matrix, a oxyamino matrix, a diamine matrix and a triamine matrix.
51. A method for purifying a lipid, a cosolvent, a surfactant or a pharmaceutical composition comprising a lipid according to aspect 49 or 50, wherein the nitrogen containing, surfactant compatible, nucleophilic matrix is selected from the group consisting of: Polymer-bound diethylenetriamine, polymer-bound p-toluene-sulfonylhydrazide and polymer-bound ethylenediamine.
52. A method for purifying a lipid, a cosolvent, a surfactant or a pharmaceutical composition comprising a lipid according to anyone of aspects 49-51, wherein the method comprises the steps of:
1) Incubation of the lipid/cosolvent/surfactant/pharmaceutical composition with a nitrogen containing, surfactant compatible, nucleophilic matrix, and
2) Isolation such as e.g. filtration, centrifugation or decantation wherein the lipid/cosolvent/surfactant/pharmaceutical composition is isolated from the nitrogen containing, surfactant compatible, nucleophilic matrix.
   53. A method for purifying a lipid, a cosolvent, a surfactant or a pharmaceutical composition comprising a lipid according to anyone of aspects 49-52, wherein the method comprises a step of passage through a column comprising a nitrogen containing, surfactant compatible, nucleophilic matrix.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

### Example 1

### Purification of propyleneglycol

Propyleneglycol (60 g) was mixed with p-toluenesulfonylhydrazide polystyrene matrix (6 g, 1% cross-linked, 100-200 mesh, substitution 1.5 mmol/g, Aldrich 532339) and the mixture was shaken gently for 20 hours. The solids were removed by either

A. filtration through polypropylene vials with polyethylene filter (MultiSynTech V200PE100). Nitrogen pressure was applied to force the liquid through the filter.
or B. centrifugation 3000 rpm for 10 minutes, followed by manual decantion.

### Generation and Purification of Lipid Mix:

The following were mixed:

30% Softigen 767 (Sasol), 40% Capmul PG 8 (Abitec) and 15% Rylo MG08 Pharma (Danisco).

The homogeneous lipid mixture (20 g) was purified on three different matrixes:

1. p-Toluenesulfonylhydrazide polystyrene matrix (2 g, g, 1% cross-linked, 100-200 mesh, substitution 1.5 mmol/g, Aldrich 532339)
2. Diethylenetriamine polystyrene matrix (2 g, 1% cross-linked, 200-400 mesh, substitution 4-5 mmol/g, Aldrich 494380)
3. Ethylenediamine matrix stratospheres (2 g, 1% cross-linked, 50-100 mesh, substitution 5-6 mmol/g, Aldrich 547484)

The solids were removed by either

A. filtration through polypropylene vials with polyethylene filter (MultiSynTech V200PE100). Nitrogen pressure was applied to force the liquid through the filter, or
B. centrifugation 3000-5000 rpm for 10 minutes, followed by manual decantion.

### Formulation of Insulin in Purified Propyleneglycol and Lipid Mixture

The derivative of insulin B29K(N(eps)Octadecanedioyl-gGlu-OEG-OEG) A14E B25H desB30 was dissolved in propyleneglycol purified according to the list in Table 1 by gently stirring 16 hours at 22° C. in closed screw cap vials flushed with nitrogen gas. Lipid mix, purified according to the list in Table 2 was added to a final sample size of 1 gr containing 25 mg insulin. The samples were gently mixed, filled on cartridges (air tight container) and closed. Chemical stability was addressed by measuring formation of the degradation products: hydrophobic impurities by RPC (reverse phase chromatography) on Waters BEH RP₈ column, 100×4.6 mm and 1.7 μm, eluted by A: 0.2 M sodium sulfate+0.04 M sodium phosphate pH 3.5+10% acetonitrile and isocratically (i) or a gradient (g) of B: 70% acetonitrile. 0-10 min i: 65/35% A/B, 10-12 min g: 44/56% A/B, 12-13 min i: 44/56% A/B, 13-15 min g: 65/35% A/B. 15-20 min i:65/35% A/B. The flow rate was 0.5 ml/min and the dual uv signal was recorded at 220 and 280 nm.

The degradation product high molecular weigth protein (HMWP) was measured by gelfiltration in 2.5 M acetic acid, 20% acetonitrile and 0.45% arginine on a Waters insulin column before and after incubation of the samples two weeks at 3TC.

Increase in degradation product formation was measured after storage 7 days at −20° and 37° C. The increase in hydrophobic related impurities and HMWP at 37° C. relatively to −20° C. are listed in Table 1 and 2.

TABLE 1
Stability of the derivative of insulin B29K(N(eps)Octadecanedioyl-
gGlu-OEG-OEG) A14E B25H desB30 in liquid lipid
for oral administration as a function of propyleneglycol
purification. The lipid components were purified
on a hydrazine matrix.

| Formulation: | |
|---|---|
| 25 g insulin | |
| 15% propyleneglycol | Chemical stability at 37° C. |
| 30% Softigen 767 | % degradation product/week |

| 40% Capmul PG 8 | | Hydrophobic |
|---|---|---|
| 15% Rylo MG08 Pharma | HMWP | related impurities |
| Hydrazide matrix | 13 | 1.82 |
| purification | | |
| Ethylenediamine | 20.3 | 1.67 |
| matrix purification | | |
| Diethylenetriamine | 13.6 | 1.51 |
| matrix purification | | |

TABLE 2
Stability the derivative of insulin B29K(N(eps)Octadecanedioyl-
gGlu-OEG-OEG) A14E B25H desB30 in liquid lipid for
oral administration as a function of lipid purification.
The propyleneglycol components were purified on
a hydrazine matrix.

| Formulation: | |
|---|---|
| 25 g insulin | |
| 15% propyleneglycol | Chemical stability at 37° C. |
| 30% Softigen 767 | % degradation product/week |

| 40% Capmul PG 8 | | Hydrophobic |
|---|---|---|
| 15% Rylo MG08 Pharma | HMWP | related impurities |
| Hydrazide matrix | 13 | 1.82 |
| purification | | |
| Ethylenediamine | 21 | 4.56 |
| matrix purification | | |
| Diethylenetriamine | 6.27 | 2.45 |
| matrix purification | | |

### Example 2

Potential aldehyde scavengers were solubilised in propyleneglycol to 1 mg/200 mg propyleneglycol. The derivative of insulin B29K A14E A21G B25H desB30 was dissolved in propyleneglycol containing scavenger according to the list in Table 3 in closed vials flushed with nitrogen gas. Capmul PG8 (Abitec) was added to 80% yielding samples of 1 gr containing 50 mg insulin and 1 mg of scavenger. The samples were gently mixed, filled on cartridges (air tight containers) and closed.

Chemical stability was addressed by measuring formation of the degradation products: deamidations and hydrophobic impurities by RPC (reverse phase chromatography) on Waters BEH RP₈ column, 100×4.6 mm and 1.7 μm, eluted by A: 0.2 M sodium sulfate+0.04 M sodium phosphate pH 3.5+10% acetonitrile and isocratically (i) or a gradient (g) of B: 70% acetonitrile. 0-10 min i: 76/24% A/B, 10-12 min g: 40/60% A/B, 12-13 min i: 40/60% A/B, 13-15 min g: 76/24% A/B. 15-20 min i:76/24% A/B. The flow rate was 0.5 ml/min and the dual uv signal was recorded at 220 and 280 nm.

The degradation product high molecular weight protein (HMWP) was measured by gelfiltration in 2.5 M acetic acid, 20% acetonitrile and 0.45% arginine on a Waters insulin column. The increase in deamidations, hydrophobic related impurities and HMWP at 37° C. relatively to −20° C. are listed in Table 3.

TABLE 3
Stability of the derivative of insulin B29K A14E A21G B25H desB30
in liquid lipid for oral administration as a function of scavenger
content. All scavengers were spraydried to pH 7.4.

| Formulation: | | | |
|---|---|---|---|
| 50 mg insulin | Chemical stability at 37° C. | | |
| 1 mg scavenger | % degradation product/week | | |
| 20% propyleneglycol | | Hydrophobic | |
| 80% Capmul PG8 | Deamidation | related impurities | HMWP |
| No scavenger | 0.9 | 2 | 1.4 |
| Glutamic acid | 1.1 | 2.4 | 1.1 |
| Ethylenediamine | 0.5 | 1 | 0.7 |
| Glutamic | 0.9 | 2.25 | 0.9 |
| acid/arginine | | | |
| Arginine | 1.25 | 2.25 | 1.6 |
| Glycylglycine | 1 | 2.2 | 0.9 |

### Example 3

The derivative of insulin B29K(N(eps)Octadecanedioyl-gGlu-OEG-OEG) A14E B25H desB30

was dissolved in propyleneglycol containing 3.3 mM ethylenediamin hydrochloride in closed vials flushed with nitrogen gas.

Equivalent lipid mixtures from three different suppliers: Abitec, Sasol, Gattefosse were added. The samples were gently mixed, filled on cartridges (air tight container) and closed. Chemical stability was addressed by measuring the degradation product high molecular weight protein (HMWP) by gelfiltration in 2.5 M acetic acid, 20% acetonitrile and 0.45% arginine on a Waters insulin column. The increase in HMWP at 37° C. relatively to −20° C. is listed in Table 4.

TABLE 4
Stability of the derivative of insulin B29K(N(eps)Octadecanedioyl-
gGlu-OEG-OEG) A14E B25H desB30 in liquid lipid for oral
administration as a function of lipid supplier.

| Formulation: | |
|---|---|
| 15% propyleneglycol | |
| 25 mg/g insulin | |
| 3.3 mM ethylenediamine- | Chemical stability at 37° C. |
| hydrochloride | % degradation product/4 weeks |
| 15% Rylo 8 | HMWP |
| Sasol/Abitec: | 4 |
| 30% Softigen 767, 40% Capmul PG8 | |
| Abitec: | 20.5 |
| 30% Acconon CC-6, 40% Capmul PG8 | |
| Gattefosse: | 1 |
| 30% Labrasol, 40% Capryol PGMC | |

### Example 4

The derivative of insulin B29K(N(eps)Octadecanedioyl-gGlu-OEG-OEG) A14E B25H desB30

was dissolved in propyleneglycol in the presence and absence of ethylenediamine hydrochloride in screw cap vials flushed with nitrogen. Lipid (30% labrasol and 40% capryol) was added by gently mixing. The samples were filled on cartridges (air tight container) and closed. Chemical stability was adressed by measuring the degradation product high molecular weigth protein (HMWP) by gelfiltration in 2.5 M acetic acid, 20% acetonitrile and 0.45% arginine on a Waters insulin column. The increase in HMWP at 37° C. relatively to −20° C. is listed in Table 5.

TABLE 5
Stability of the derivative of insulin B29K(N(eps)Octadecanedioyl-
gGlu-OEG-OEG) A14E B25H desB30 in liquid lipid for
oral administration as a function of presence of
ethylenediamine hydrochloride

| Formulation: | |
|---|---|
| 15% propyleneglycol | |
| 25 mg/g insulin | |
| 15% Rylo MG08 Pharma | Chemical stability at 37° C. |
| 30% Labrasol | % degradation product/4 weeks |
| 40% Capryol PGMG | HMWP |
| 3.3 mM ethylenediamine hydrochloride | 1 |
| in the propyleneglycol | |
| 0 mM ethylenediamine hydrochloride | 3 |
| in the propyleneglycol | |

### Example 5

Lipid purification, the following lipid mixtures were prepared:

### 1. 15% Rylo MG08, 30% Acconon CC6, 40% Capryol PGMG

### 2. 15% Rylo MG08, 30% Labrasol, 40% Capryol PGMG.

### 3. 15% Rylo MG08, 30% Labrasol, 40% Capmul PG8.

The lipid mixtures (20 g) were each purified on diethylenetriamine polystyrene matrix (2 g, 1% cross-linked, 200-400 mesh, substitution 4-5 mmol/g, Aldrich 494380)

The matrix was removed by filtration through polypropylene vials with polyethylene filter (MultiSynTech V200PE100). Nitrogen pressure was applied to force the liquid through the filter.

The derivative of insulin B29K(N(eps)Octadecanedioyl-gGlu-OEG-OEG) A14E B25H desB30

was dissolved in propyleneglycol and mixed with lipid according to Table 6. Purified and unpurified lipid mixtures were added, the samples were gently mixed the samples were filled on cartridges (air tight containers) and closed.

TABLE 6
Stability of the derivative of insulin B29K(N(eps)Octadecanedioyl-
gGlu-OEG-OEG) A14E B25H desB30 in liquid lipid
for oral administration as a function of purification
of lipid mix.

| Formulation: | Chemical stability at 37° C. |
|---|---|
| 15% propyleneglycol | % degradation product/4 weeks |
| 25 mg/g insulin | HMWP |
| Purified: | 2 |
| 15% Rylo MG08 Pharma | |
| 30% Labrasol | |
| 40% Capryol PGMG | |
| Non-purified: | 3 |
| 15% Rylo MG08 Pharma | |
| 30% Labrasol | |
| 40% Capryol PGMG | |
| Purified: | 12 |
| 15% Rylo MG08 Pharma | |
| 30% Acconon CC-6 | |
| 40% Capmul PG8 | |
| Non-purified: | 20.5 |
| 15% Rylo MG08 Pharma | |
| 30% Acconon CC-6 | |
| 40% Capmul PG8 | |
| Purified: | 8 |
| 15% Rylo MG08 Pharma | |
| 30% Acconon CC-6 | |
| 40% Capryol PGMG | |
| Non-purified: | 11 |
| 15% Rylo MG08 Pharma | |
| 30% Acconon CC-6 | |
| 40% Capryol PGMG | |

### Example 6

### Labrasol Treatment Optimized for Matrix Ratio, Temperature and Time

Labrasol was treated with diethylenetriamine polystyrene matrix, 1, 5 or 10% w/w at 30, 40 or 50° C. for 2, 4, 6 or 16 hours.

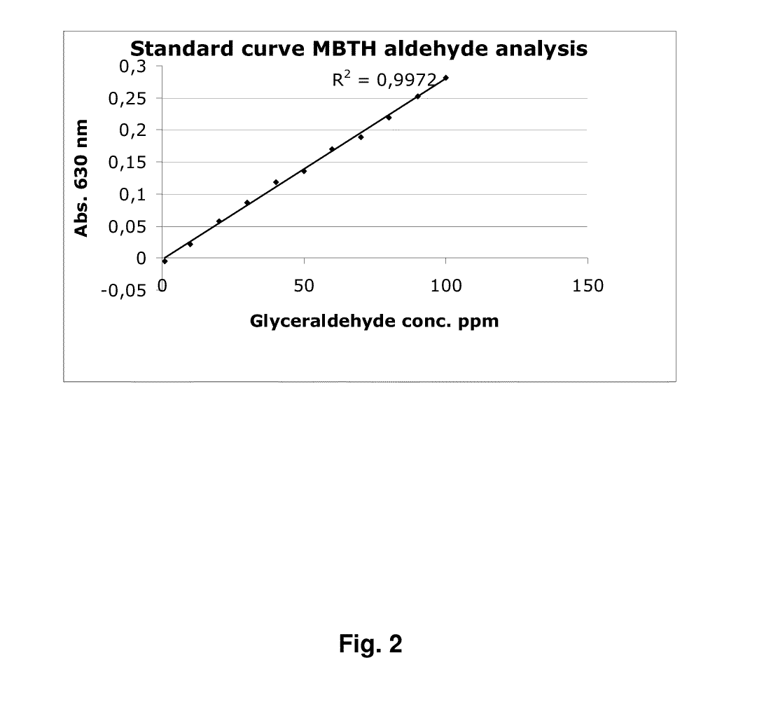

NMR analysis showed that all aldehyde was removed from labrasol when using as minimum 5% resin w/w, 40° C., 16 hours or 10% resin w/w 40° C., 6 hours (FIG. 2).

### Example 7

### Colorimetric Measurement of Aldehyde in Propylene Glycol

MBTH solution: 3-methyl-2-benzothiazolinone hydrazone.HCl.H₂O (50 mg) was dissolved in water (100 mL), stored in amber flask at 5° C. for a week at the most.

Ferric chloride solution: Ferric chloride (30 g) and conc. hydrochloric acid was dissolved in water (100 ml). This solution (5.4 g) was mixed with Sulfamic acid (1.5 g) and diluted with water to 100 mL.

Propylene glycol sample (50 mg) was mixed with MBTH solution (2 mL), ferric chloride solution (2 mL) and water (0.5 mL) and heated on boiling water batch 1 minute. After about 30 minutes the UV/Vis absorbtion was measured at 620 nm (blue color) against a blank sample (2 mL MBTH solution+2.5 mL ferric chloride solution+0.5 mL water).

Reference Anal. Chem. 1964, 36, 3.

A standard curve was prepared with DL-glyceraldehyde 0-100 ppm in water.

TABLE 7

| | UV 630 | aldehyde, |
|---|---|---|
| Propylene glycol sample | nm | ppm |
| VSCK 300 Pharm | 0.01 | <1 |
| VSCK 300 + 50 ppm glyceraldehyde spike | 0.166 | 52 |
| VWR 0509534 | 0.038 | <2 |
| SSCY 566 Merck | 0.037 | <2 |
| NNTSCSO87 | 0.014 | <1 |
| Catalent OET-00304185, Dow chem XH2401A510 | 0.003 | <1 |
| Sigma 068K0068 | 0.003 | <1 |

### Example 8

The following excipients were purified and analysed for aldehyde content, as described in examples 1 and 6. Specifically, the Labrasol ALF and diglycerol caprylate were both treated with 10% (w/w) diethylenetriamine resin for 20 hours at 25° C., while Rylo MG08 was treated at 55° C. For the purification of the propylene glycol 10% (w/w) p-toluene-sulfonylhydrazine resin was used in stead of the diethylenetriamine resin. The purified excipients were used in the formulations shown in table 8.

Extraction method: The SMEDD formulations were allowed to reach room temperature. To 20 μl of the SMEDD formulation, 490 μl 1-butanol was added followed by addition of 990 μl of 0.1% (w/w) Tween80, 0.1M Na₂HPO₄/NaH₂PO₄ pH 7.0. The formulations were than vortexed and incubated at RT for 30 min followed by vortex again and then centrifugation at RT at 14000 rpm for 20 min. The bottom water phase was analysed.

Chemical stability was assessed by analysis of the water phase on RPC (reverse phase chromatography) using a Waters BEH Shield RP18 UPLC column (2.1×100 mm, 1.7 μm) and the following run parameters:

Wavelength: 215 nm, Column temperature: 50° C., Flow: 0.4 ml/min, Run time: 18.5 min, Sample load: 7.5 μl, Buffer A: 0.09M di-ammonium hydrogen phosphate pH 3.0, 10% acetonitrile, Buffer B: 90% acetonitrile.

TABLE 8
RPC method used for the analysis of chemical stability

| Time (min) | Flow (ml/min) | % A | % B | Curve |
|---|---|---|---|---|
| Initial | 0.400 | 73.0 | 27.0 | |
| 1.00 | 0.400 | 73.0 | 27.0 | 11 |
| 2.50 | 0.400 | 68.0 | 32.0 | 6 |
| 12.00 | 0.400 | 50.0 | 50.0 | 6 |
| 13.50 | 0.400 | 20.0 | 80.0 | 6 |
| 15.00 | 0.400 | 20.0 | 80.0 | 6 |
| 17.00 | 0.400 | 73.0 | 27.0 | 6 |
| 19.00 | End | End | End | 11 |

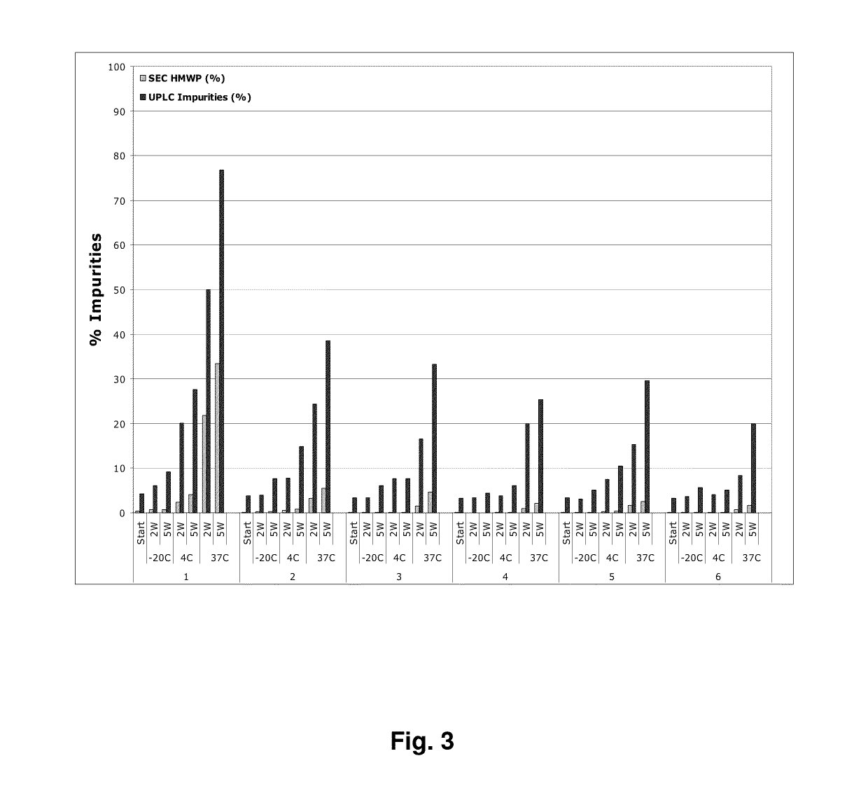

Furthermore, the samples were analysed by SEC (size exclusion chromatography) in 2.5 M acetic acid, 20% acetonitrile and 0.45% arginine on a Waters insulin column for the degradation product high molecular weight protein (HMWP). The results are shown in FIG. 3.

TABLE 9
Contents of the formulations:
All have 25 mg/g derivative of insulin

1. Non-purified excipients:
15% Propylene glycol
20% Labrasol ALF
30% Super refined polysorbate 20 (Croda)
35% Diglycerol caprylate
2. Non-purified excipients + scavenger:
1 mg/ml ethylene diamine
15% Propylene glycol
20% Labrasol ALF
30% Super refined polysorbate 20 (Croda)
35% Diglycerol caprylate
3. Purified excipients:
15% Propylene glycol
20% Labrasol ALF
30% Super refined polysorbate 20 (Croda)
35% Diglycerol caprylate
4. Purified excipients + scavenger:
1 mg/ml ethylene diamine
15% Propylene glycol
20% Labrasol ALF
30% Super refined polysorbate 20 (Croda)
35% Diglycerol caprylate
5. Non-purified excipients + scavenger:
1 mg/ml ethylene diamine
15% Propylene glycol
40% Labrasol ALF
45% Rylo MG08
6. Purified excipients:
15% Propylene glycol
40% Labrasol ALF
45% Rylo MG08

### Example 9

The derivative of insulin B29K(N(eps)Octadecanedioyl-gGlu-OEG-OEG) A14E B25H desB30 was dissolved in propyleneglycol in the presence and absence of ethylenediamine hydrochloride in screw cap vials flushed with nitrogen. Lipid (40% labrasol and 45% Rylo MG08 Pharma) was added by gently mixing. The samples were filled on cartridges (air tight container) and closed. Chemical stability was addressed by measuring the degradation product high molecular weight protein (HMWP) by gelfiltration in 2.5 M acetic acid, 20% acetonitrile and 0.45% arginine on a Waters insulin column. The increase in HMWP at 37° C. relatively to −20° C. is listed in Table 10.

TABLE 10
Stability of the derivative of insulin B29K(N(eps)Octadecanedioyl-
gGlu-OEG-OEG) A14E B25H desB30 in liquid lipid for
oral administration as a function of presence of
ethylenediamine hydrochloride

| Formulation: | |
|---|---|
| 15% propyleneglycol | |
| 25 mg/g insulin | Chemical stability at 37° C. |
| 15% Rylo MG08 Pharma | % degradation product/4 weeks |
| 30% Labrasol | HMWP |
| 3.3 mM ethylenediamine | 1.5 |
| hydrochloride in the propyleneglycol | |
| 1.65 mM ethylenediamine | 2.5 |
| hydrochloride in the propyleneglycol | |
| 0.8 mM ethylenediamine | 3 |
| hydrochloride in the propyleneglycol | |
| 0 mM ethylenediamine | 25 |
| hydrochloride in the propyleneglycol | |

### Example 10

Three sources of propylene glycol were tested by dissolving a derivative of insulin in the propylene glycol at a concentration of 25 mg/g insulin: Propylene glycol A (Merck), propylene glycol B (Sigma Aldrich P4347), and propylene glycol C (Dow Chemical Company, purity>99.8%).

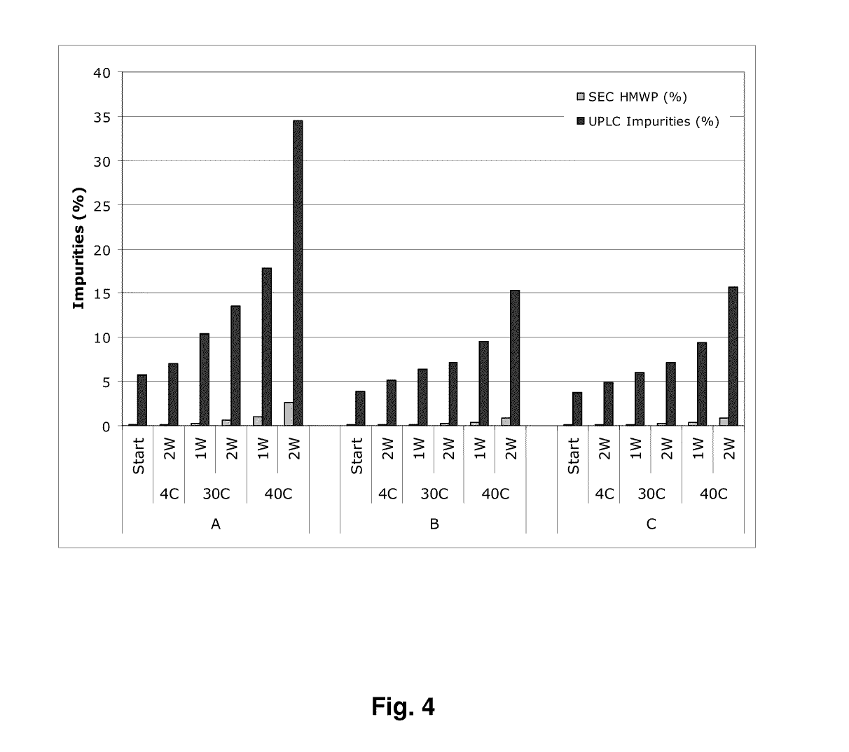

The extraction method used was as described in example 10. Chemical stability was assessed by analysis of the water phase on RPC (reverse phase chromatography) using a Waters BEH Shield RP18 UPLC column (2.1×100 mm, 1.7 μm) as described in example 10. Furthermore, the samples were analysed by SEC (size exclusion chromatography) in 2.5 M acetic acid, 20% acetonitrile and 0.45% arginine on a Waters insulin column for the degradation product high molecular weight protein (HMWP). The results are shown in FIG. 4.

### Example 11

Two sources of Labrasol from Gattefossé and one batch of purified Labrasol were tested by dissolving a derivative of insulin in the propylene glycol at a concentration of 25 mg/g insulin, and then adding Labrasol. The final formulations were all of the form: 25 mg/g derivative of insulin, 50% Propylene glycol, 50% Labrasol. The Labrasols used was: No. 1: Labrasol, technical grade from Gattefossé, no. 2: Labrasol ALF phama grade from Gattefossé, and no. 3: Labrasol ALF purified as described in example 6. The aldehyde content of the purified Labrasol ALF was measured by NMR as described in example 15, and NMR spectra are shown in FIG. 2.

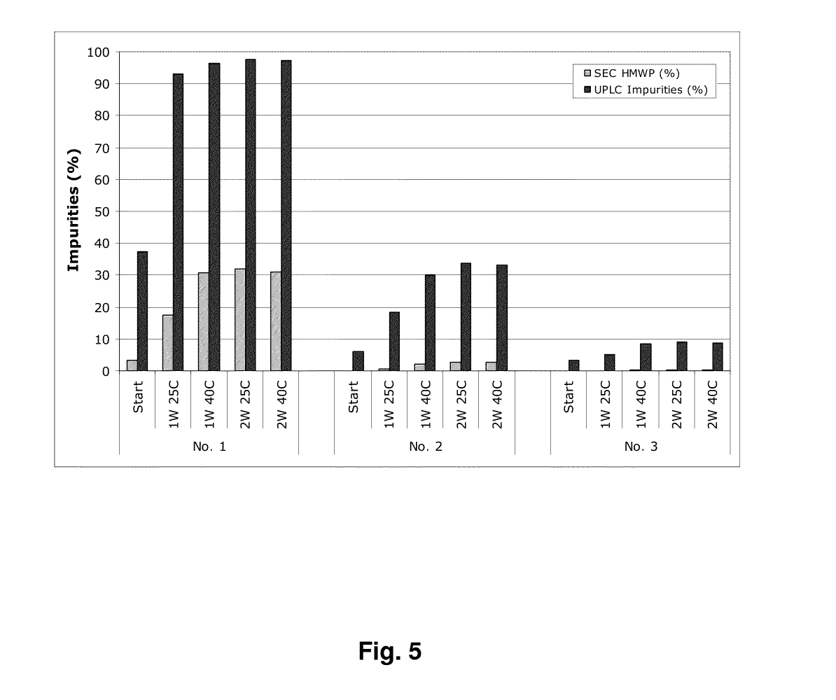

The extraction method used was as described in example 10. Chemical stability was assessed by analysis of the water phase on RPC (reverse phase chromatography) using a Waters BEH Shield RP18 UPLC column (2.1×100 mm, 1.7 μm) as described in example 10. Furthermore, the samples were analysed by SEC (size exclusion chromatography) in 500 mM NaCl, 10 mM NaH₂PO₄, 5 mM H₃PO₄, 50% (v/v) 2-propanol on a Waters insulin column for the degradation product high molecular weight protein (HMWP). The results are shown in FIG. 5.

### Example 12

### NMR Based Measurement of Aldehyde Content

In order to obtain a spectrum where the signal intensity is primarily dependent on the spectrometer sensitivity and the amount of aldehyde present, all NMR signals arising from regions outside of a 4 ppm spectral window centred around 9 ppm were suppressed using a double pulsed field gradient spin echo experiment incorporating band selective inversions. In order to maximise the robustness of the method with regards pulse miscalibration and quantification errors the constant adiabaticity Gaussian inversion pulses were used for the selective inversion. In order to avoid the issues related to miscibility of the substances under investigation all spectra were acquired with either an external lock substance in a coaxial insert or unlocked using drift compensation. Given the additional signal due to the extra sample in the active volume of spectrometer probe head, the unlocked method is the preferred method unless the spectrometer is not sufficiently shielded from external perturbations of the Bₒ-field

1. A non-aqueous liquid pharmaceutical composition comprising at least one lipid, at least one insulin, at least one scavenger and optionally at least one surfactant, wherein the scavenger is a nitrogen containing nucleophilic compound.

2. A non-aqueous liquid pharmaceutical composition according to claim 1, wherein the scavenger is ethylenediamine or a derivative thereof.

3. A non-aqueous liquid pharmaceutical composition according to claim 1, wherein the lipid and/or surfactant is a high purity lipid.

4. A non-aqueous liquid pharmaceutical composition according to claim 1, wherein the lipid and/or surfactant has an aldehyde and/or ketone content below 20 ppm when added to the pharmaceutical composition.

5. A non-aqueous liquid pharmaceutical composition according to claim 1, wherein the lipid and/or surfactant has been purified using a nitrogen containing, surfactant compatible, nucleophilic matrix before being added to the pharmaceutical composition.

6. A non-aqueous liquid pharmaceutical composition according to claim 1, wherein the surfactant is a non-ionic surfactant.

7. A non-aqueous liquid pharmaceutical composition according to claim 1, wherein the lipid and/or surfactant is selected from the group consisting of: Glycerol mono-caprylate (such as e.g. Rylo MG08 Pharma), Glycerol mono-caprate (such as e.g. Rylo MG10 Pharma from Danisco), polyglycerol fatty acid ester (such as e.g. Plurol Oleique or Diglycerol monocaprylate), caprylocaproyl macrogol-8-glycerides (such as e.g. Labrasol ALF), polysorbate 20 (such as Tween 20 or super refined Tween 20) and polysorbate 80 (such as Tween 80 or super refined Tween 80).

8. A non-aqueous liquid pharmaceutical composition according to claim 1 further comprising a cosolvent, which is propylene glycol.

9. A non-aqueous liquid pharmaceutical composition according to claim 1, wherein the insulin is a derivative of insulin.

10. A method for manufacturing a non-aqueous liquid pharmaceutical composition according to claim 1.

11. A method for manufacturing a non-aqueous liquid pharmaceutical compositioncomprising at least one lipid, at least one insulin, and a cosolvent, wherein said cosolvent, said lipid and said optional surfactant are first purified on a nitrogen containing, surfactant compatible, nucleophilic matrix before being added to the composition.

12. A method for manufacturing a non-aqueous liquid pharmaceutical composition according to claim 11, wherein the pharmaceutical composition comprises a cosolvent and a scavenger, wherein the scavenger is dissolved in said purified cosolvent as a first step of the method of manufacturing the pharmaceutical composition, then, as a second step, the insulin is dissolved in the scavenger containing cosolvent.

13. A method for manufacturing a non-aqueous liquid pharmaceutical composition according to claim 12, wherein the scavenger is neutralized before being dissolved in said cosolvent.

14. A method for manufacturing a non-aqueous liquid pharmaceutical composition according to claim 12, wherein the insulin is dissolved by gentle stirring in a mixture comprising ethylenediamine and propylene glycol.

15. A method for purifying a lipid, a cosolvent, a surfactant or a pharmaceutical composition comprising a lipid, wherein purification is performed on a nitrogen containing, surfactant compatible, nucleophilic matrix, whereby removal of excess aldehyde is achieved.
